(19) United States
(12) Patent Application Publication
(10) Pub. No.: US 2023/0105889 A1
(43) Pub. Date: Apr. 6, 2023

(54) IRRIGATION CATHETER WITH OPTIONAL ENTERAL FEEDING FUNCTIONALITY OR STOMACH-IN FLUID ASPIRATION FUNCTIONALITY

(71) Applicant: MICRO-TECH ENDOSCOPY USA, INC., Ann Arbor, MI (US)

(72) Inventors: Kenneth F. BINMOELLER, San Diego, CA (US); John Lunsford, Los Altos Hills, CA (US); Fiona Sander, Los Altos Hills, CA (US); Hoang Phan Milpitas, Ann Arbor, MI (US)

(21) Appl. No.: 17/934,438

(22) Filed: Sep. 22, 2022

Related U.S. Application Data
(60) Provisional application No. 63/247,152, filed on Sep. 22, 2021.

(51) Int. Cl. A61J 15/00; A61B 17/34

(52) U.S. Cl. CPC A61J 15/0015; A61B 17/3415

(57) ABSTRACT

A gastrointestinal catheter for insertion into a treatment site in a patient, the treatment site including at least one of a first treatment site and a second treatment site, where the catheter includes at least one of a first and a second treatment member; the proximal portions of the first and second treatment members may be detachably or slidably conjoinable side by side; the distal portions of the first and second treatment members are separate from each other. The distal end of the first treatment member is configured for insertion into the first treatment site; the second treatment member is configured to be placed at a second treatment site. The distal end region of the first treatment member has an anchoring configuration adapted to anchor the distal end region within the first treatment site, the distal end region is further capable of elastically assuming a linear constrained configuration.

### CROSS-REFERENCE TO RELATED APPLICATION(S)

This application claims priority to U.S. Provisional Pat. Application No. 63/247,152, filed on Sep. 22, 2021, which is hereby incorporated by reference in its entirety.

### FIELD

Embodiments of the disclosure are directed to interventional gastroenterology devices. Particular embodiments relate to catheters adapted to irrigate walled off necrotic sites as well as, in some embodiments, adapted to provide a route for enteral feeding or stomach-in fluid aspiration.

### BACKGROUND

Patients suffering from acute necrotizing conditions such as pancreatitis can develop walled-off necrosis, filled with fluid and cellular debris, that can be cleared with interventional irrigation or flushing treatment. In medical literature, these sites may be referred to by the acronym “WON” (walled-off necrosis) or the acronym “WOPN” (walled-off pancreatic necrosis) when specifically applicable to the pancreas. Earlier terminology has referred to these pancreatic sites as a pancreatic absess or an infected pancreatic pseudocyst. Endoscopic drainage and flushing of the walled-off site is commonly performed with catheters designed for other purposes, such as use of the Cook nasal biliary drainage catheter that is actually designed for draining the bile duct.

Embodiments of the present disclosure are directed toward an irrigation catheter having features that are adapted for the anatomy of a walled-off necrotic site, and appropriate for the anatomy of the path by which the catheter approaches and is withdrawn from a targeted treatment site.

### SUMMARY

The present disclosure provides a gastrointestinal catheter, a gastrointestinal catheter kit and a method of flushing a first treatment site with the same, and a method of treating treatment sites in a gastrointestinal tract with the same.

In a first aspect of the present disclosure, there is provided a gastrointestinal catheter, the gastrointestinal catheter for insertion into a treatment site in a patient, the site in need of medical treatment, the treatment site comprising at least one of a first treatment site and a second treatment site, the catheter comprising: a treatment member, the treatment member comprising at least one of a first treatment member and a second treatment member and comprising a lumen, a proximal portion, a distal portion, and a distal end region within the distal portion; wherein the first treatment member is sized and configured for insertion of its distal end into the first treatment site in an upper portion of a gastrointestinal tract, and wherein the second treatment member is sized and configured for placement of its distal end into the second treatment site within a bowel or a stomach of the patient; wherein the distal end region of the first treatment member comprises an anchoring configuration, the treatment member being capable of assuming a linear constrained configuration, wherein the anchoring configuration is adapted to anchor the distal end region within the first treatment site; wherein the distal end region of the first treatment member comprises one or more fluid ports in communication with the lumen, wherein the fluid ports are configured to allow through flow of a fluid; wherein the proximal portions of the first and second treatment members are detachably or slidably conjoinable side by side, and wherein the distal portions of the first and second treatment members are separated from each other.

In a second aspect of the present disclosure, there is provided a gastrointestinal catheter kit, the gastrointestinal catheter kit comprises the gastrointestinal catheter of the first aspect; and a pusher member sized and configured to fit within the endoscope, the pusher member comprising a lumen, wherein the pusher member is configured to stabilize a position of the gastrointestinal catheter in at least one of the first treatment site and the second treatment site.

In a third aspect of the present disclosure, there is provided a method of flushing a first treatment site with the gastrointestinal catheter of the first aspect, comprising: placing a percutaneous endoscopic gastrostomy (PEG) tube in the patient thereby providing an entry route for the at least one of the first treatment member and the second treatment member into a gastrointestinal tract of the patient; advancing the second treatment member distally to a second treatment site in a small bowel or a stomach of the patient; advancing the first treatment member over a guidewire to a position such that a distal end region of the first treatment member is positioned within a first treatment site; withdrawing the guidewire proximally from the first treatment member, thereby anchoring the distal end region of the first treatment member within the first treatment site; flowing an irrigation fluid through the treatment member and into the treatment site to flush the first treatment site; and flowing a solution through the second treatment member to the second treatment site to conduct enteral feeding or aspirating a fluid at the second treatment site through the second treatment member.

In a fourth aspect of the present disclosure, there is provided a method of treating treatment sites in a gastrointestinal tract with the gastrointestinal catheter of the first aspect, the method comprising: placing a percutaneous endoscopic gastrostomy (PEG) tube in a patient thereby providing an entry route for the double lumen gastrointestinal catheter into a gastrointestinal tract of the patient, the catheter comprising (1) a first treatment member comprising a first lumen and (2) a second treatment member comprising a second lumen; distally advancing the second treatment member into a second treatment site in a small bowel or a stomach of the patient; distally advancing the first treatment member to a position such that a distal end region of the first treatment member is positioned within a first treatment site; anchoring the distal end region of the first treatment member within the first treatment site; flowing an irrigation fluid through the first treatment member and into the first treatment site; and flowing a solution through the second treatment member into the second treatment site or aspirating a fluid at the second treatment site through the second treatment member.

### BRIEF DESCRIPTION OF THE FIGURES

FIG. 1 shows an embodiment of a single lumen gastrointestinal catheter sized and configured for flushing a treatment site in the gastrointestinal tract, such as a necrotic site involving the pancreas.

FIG. 2 shows an embodiment of an assistive pusher member 52 that may be used in conjunction with a gastrointestinal catheter to stabilize the position of the gastrointestinal catheter in a treatment site.

FIG. 3 shows an embodiment of a double lumen gastrointestinal catheter 410 placed within a patient by percutaneous access, the catheter having both treatment site flushing capability and small bowel site enteral feeding capability or stomach-in fluid aspiration capability.

FIGS. 4A-4E show various fluid port embodiments that may be disposed in a distal end region of a gastrointestinal catheter configured for flushing a treatment site. FIG. 4A has fluid ports configured as holes, FIG. 4B has fluid ports configured as slits, FIG. 4C has fluid ports configured as skives, FIG. 4D has fluid ports configured as spray jets or drip sites, and FIG. 4E has a fluid port configured as a single large hole.

FIG. 5 shows a cross sectional view of a large fluid port 40e within the distal portion of a gastrointestinal catheter 10 configured for flushing a treatment site.

FIGS. 6A-6B show views of fluid ports within the distal tip of a gastrointestinal catheter embodiment configured for flushing a treatment site. FIG. 6A shows a side view of the distal portion of a catheter that has a single loop. FIG. 6B shows a cross sectional perspective view of the distal end loop, the lumen of the catheter hosting a guidewire.

FIGS. 7A-7C show various views of the distal end of an embodiment of a gastrointestinal catheter configured for flushing a treatment site, this particular embodiment having a distal anchoring end configured as a double loop as its preferred configuration. FIG. 7A shows a side view, FIG. 7B shows a top view, and FIG. 7C shows a face end view.

FIGS. 8A-8B show of embodiments of a tip for the distal end of a gastrointestinal catheter configured for flushing a treatment site. FIG. 8A shows a tapered tip with a distal orifice; FIG. 8B shows a bullet-shaped tip with a closed end.

FIG. 9 shows a side view of an embodiment of double lumen gastrointestinal catheter configured for percutaneous access, the catheter including a first treatment member for flushing a treatment site and a second treatment member for enteral feeding into the small bowel or for aspirating fluid in the stomach.

FIG. 10 shows a view of an embodiment of a double lumen gastrointestinal catheter configured for percutaneous access, the catheter having an anchor in the form of two balloon configured to stabilize the catheter at the percutaneous access site.

FIG. 11 shows a view of an embodiment of a double lumen gastrointestinal catheter configured for percutaneous access, the catheter having an anchor in the form of a single balloon configured to stabilize the catheter at the percutaneous access site.

FIG. 12 shows a view of an embodiment of a double lumen gastrointestinal catheter configured for percutaneous access, the catheter having an anchor in the form of flexible skirt anchor configured to stabilize the catheter at the percutaneous access site.

FIG. 13 shows a perspective view of an embodiment of an anchoring bolster with a friction-locking feature, the bolster configured to stabilize a double lumen gastrointestinal catheter configured for percutaneous access.

FIGS. 14A-14B show an embodiment of an anchoring bolster with a locking thread feature, the bolster configured to stabilize a double lumen gastrointestinal catheter configured for percutaneous access. FIG. 14A is a perspective view of a bolster with a threaded connection feature; FIG. 14B is a cross sectional view of the threaded portion of the threaded bolster.

FIG. 15 shows a side view of an embodiment of a second treatment member of a double lumen gastrointestinal catheter configured for percutaneous access, the second treatment member configured for enteral feeding into the small bowel or for aspirating fluid in the stomach.

FIG. 16 shows an embodiment of a transitional region of a gastrointestinal catheter member where proximal and distal portions meet, and wherein the stiffness of the distal portion is greater than that of the proximal portion, the variable stiffness provided by variation in thickness of the sidewall.

FIGS. 17A-17C show views of a transitional region of a gastrointestinal catheter member where proximal and distal portions meet, and wherein the stiffness of the distal portion is greater than that of the proximal portion, the variable stiffness provided by orthogonally aligned ridging features within the sidewall. FIG. 17A shows a side view, FIG. 17B shows a perspective view, and FIG. 17C shows a cross sectional view.

FIGS. 18A-18C show views of a transitional region of a gastrointestinal catheter member where proximal and distal portions meet, and wherein the stiffness of the distal portion is greater than that of the proximal portion, the variable stiffness provided by sidewall axially-arranged convolutional features. FIG. 18A shows a side view of the catheter, FIG. 18B shows a cross sectional view of a proximal portion of the catheter, and FIG. 18C shows a cross sectional view of a distal portion of the catheter.

FIG. 19 shows views of an intersectional region of a gastrointestinal catheter where proximal and distal portions meet, and wherein the stiffness of the distal portion is greater than that of the proximal portion, the variable stiffness provided by differences in layered construction of the sidewall.

FIG. 20 shows the flow paths for irrigation fluid through and out a distal end region of gastrointestinal catheter, the distal end disposed within a treatment site in need of flushing.

FIG. 21 shows a side view of an embodiment of a double lumen gastrointestinal catheter, the catheter having a first treatment member with a treatment site irrigation capability and a second treatment member with a small bowel enteral feeding capability or a stomach-in fluid aspiration capability.

FIG. 22 shows one of the treatment members of an embodiment of a double lumen gastrointestinal catheter having a dovetail joint that connects the two lumens in a conjoined proximal portion of the catheter, the distal portions of the two members being separate distal to the conjoined section, the lumen of each member being hemi-circular in the proximal conjoined region and fully circular in the distal end region.

FIG. 23 shows an embodiment of a double lumen gastrointestinal catheter as placed within a patient, the catheter having a first member configured for treatment site flushing capability and a second member configured for small bowel enteral feeding capability or stomach-in fluid aspiration capability.

FIGS. 24A-24B show cross-sectional views of a dovetail joint in a conjoined section of an embodiment of a double lumen gastrointestinal catheter. FIG. 24A shows the two luminal portions disengaged; FIG. 24B shows the two luminal portions joined together by the dovetail joint.

FIG. 25 shows a cross-sectional view of a conjoined section of an embodiment of a double lumen gastrointestinal catheter, the two lumens joined by an outer layer sheath.

FIG. 26A shows a perspective view of an embodiment of a double lumen gastrointestinal catheter configured for percutaneous access, and FIG. 26B shows a side view of an embodiment of a double lumen gastrointestinal catheter configured for percutaneous access, the catheter including a connection member and sealing caps.

FIGS. 27A-27C show views of various embodiments of a connection member.

FIG. 28A shows a side view of an embodiment of a straight rectifying tube, FIG. 28B shows a cross-sectional view of an embodiment of a straight rectifying tube.

### DETAILED DESCRIPTION

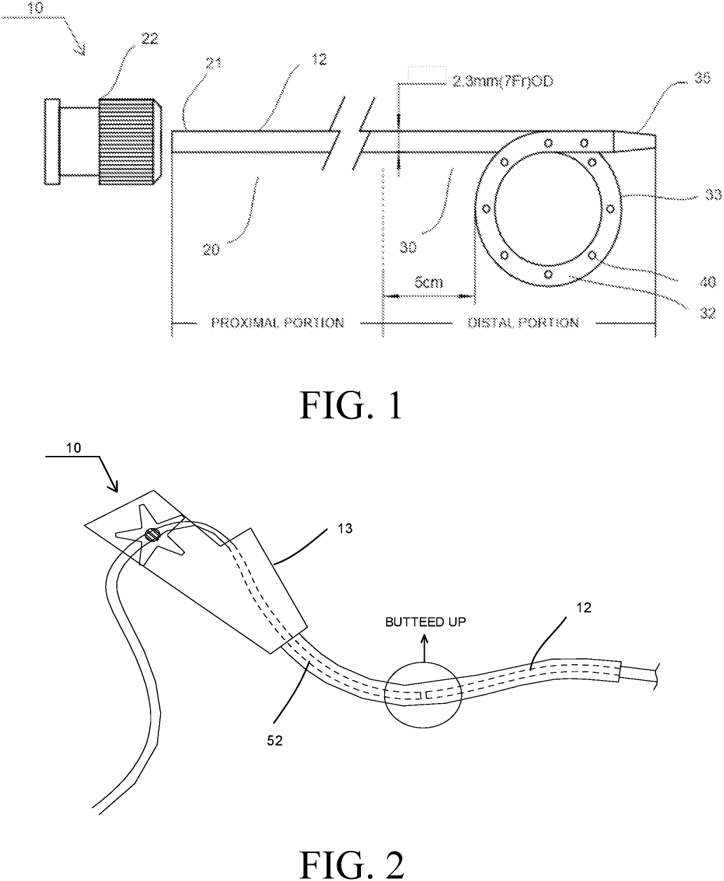

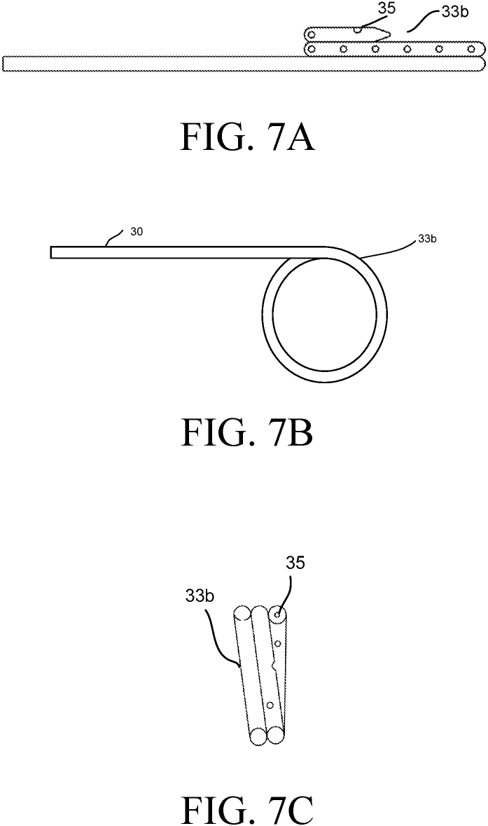

FIG. 1 shows an embodiment of a single lumen gastrointestinal catheter 10 sized and configured for flushing a treatment site associated with, near, or easily accessible from the gastrointestinal tract, such as pancreatic walled-off necrosis. Embodiments of gastrointestinal catheter 10 include a treatment member 12 that has a proximal portion 20, a distal portion 30, and a distal end region 32 within the distal portion. Embodiments of the distal end region 32 of distal portion 30 typically have a preferred configuration in the form of a loop, as shown in various embodiments of the disclosure in FIGS. 1, 6A, 9, 20, 21, and 23. In an alternative preferred configuration, embodiments of distal end region may take the form of a two or more loops, as shown in FIGS. 7A-7C. FIG. 1 further shows fluid ports 40 disposed within distal end region 32; the fluid ports are disposed within the distal end region such that when the distal end region is disposed within a treatment site, the fluid ports are positioned entirely within the treatment site. A proximal end 21 of the treatment member 12 may connect with one end of an adapter 22, and the other end of the adapter 22 is configured to be fitted with other device, for example, a syringe, when performing, for example, suction and injection functions. The adapter 22 may be female luer compression fitting.

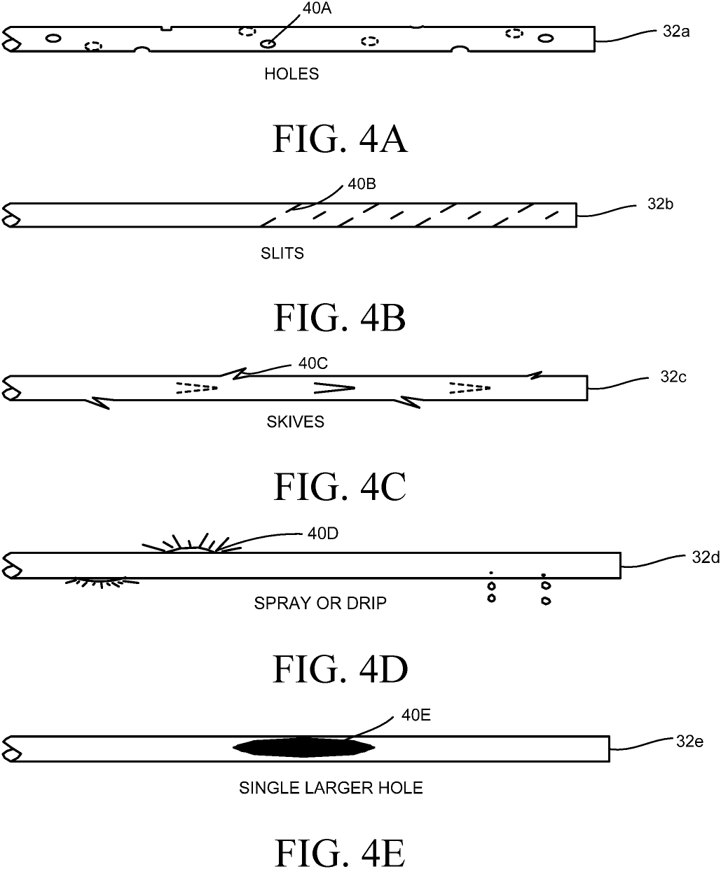

The preferred configuration of distal end region 30, as exemplified by a single loop 33a or multiple loops 33b (FIGS. 7A-7C) serves to anchor the distal end region within a treatment site, which is typically an anatomical cavity. In the absence of sufficient stiffness, the distal end region could slip out of the treatment site during a course of medical treatment. Further description of anchoring loop embodiments 33a and 33b is provided in context FIGS. 4A-4E, 5, 6A-6B, and 7A-7C.

Embodiments of treatment member 12 typically have a length on the order of about 230 cm, but may range in length from about 50 cm to about 350 cm, and a typical outer diameter (OD) of about 2.3 mm (7 French), but may range from about 1.66 mm to about 3.66 mm (5-11 French). Embodiments of the proximal portion 20 are configured to accept a fitting that will connect with an irrigation fluid source. The internal diameter (ID) of treatment member 12 is sized to accept a guidewire (having a diameter of about 0.035 in to about 0.038 in) that can be used to facilitate endoscopic placement.

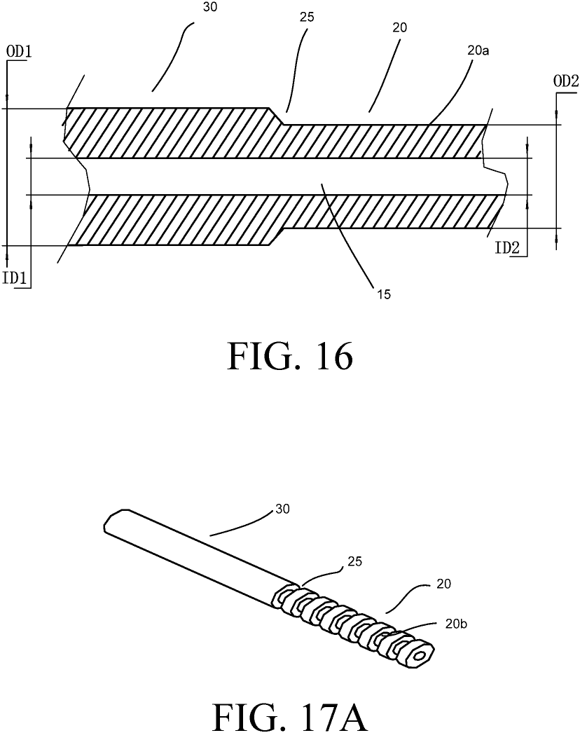

Distal portion 30 and proximal portion 20 differ from each other with regard to stiffness, the stiffness of distal portion 30 being higher than that of proximal portion 20. The relative stiffness of distal portion 30, and particularly of distal end region 32 is advantageous in that the stiffness allows the distal end region to maintain its preferred anchoring configuration, as noted above. The relative softness or compliance of proximal portion 30 of treatment member 12 is advantageous in that such compliance is friendly to the patient, mitigating the discomfort that can be associated with its presence in the nose and as it emerges externally from the body and contacts or comes to be wrapped around the patient’s face, head, or neck. As soft or compliant as proximal portion 30 may be, it still needs to be stiff enough to resist kinking or collapse. Physical or structural aspects of proximal port 20 and distal portion 30 that underlie differing stiffness are described further below in the context of FIGS. 16-19.

Stiffness is commonly characterized in terms of the modulus of elasticity; the distal portion of the treatment member of embodiments of gastrointestinal catheter has a higher modulus of elasticity than that of the proximal portion. Regardless of the particular value of elastic modulus of the distal portion of the treatment member, it is sufficiently high that upon insertion into the treatment site and release from a substantially linear constrained configuration, the distal end region assumes its preferred anchoring configuration.

In various embodiments, the distal portion of the treatment member may have an elastic modulus in the range of about 40,000 psi to about 600,000 psi, in the range of about 40,000 psi to about 300,000 psi, in the range 40,000 psi to about 150,000 psi, or in the range of about 40,000 psi to about 75,000 psi. By way of example, a product of Arkema provides a polyether block amide product, PEBAX® 6333, having an elastic modulus of 41,300 psi.

The elastic modulus ranges of various embodiments of the distal portion of the gastrointestinal catheter do not necessarily exclude embodiments of the proximal portion from those ranges so long as the elastic modulus of the proximal portion is less than that of the distal portion. Accordingly, when not overlapping with the elastic modulus of the distal portion, the proximal portion of the treatment member may have an elastic modulus in the range of about 1,000 psi to about 75,000 psi or in the range of about 1,500 psi to about 42,000 psi. In some embodiments, the proximal portion of the treatment member comprises an elastic modulus in the range of about 1,700 psi to about 25,000 psi. By way of example, a product of Arkema provides a polyether block amide product, PEBAX® 2533, having an elastic modulus of 1,750 psi and PEBAX® 633having an elastic modulus of 41,300 psi.

FIG. 2 shows an embodiment of an assistive pusher member 52 that may be used in conjunction with a gastrointestinal catheter 10 to stabilize the position of the gastrointestinal catheter in a treatment site, as described in the context of provided methods of operating the disclosure. The use of the pusher member 52 in stabilizing the position of a distal end region of a gastrointestinal catheter in a treatment site is described elsewhere in the context of method embodiments of the disclosure. In an embodiment of the present disclosure, a treatment member 12 may be advanced into a patient and to a treatment site by the pusher member 52, in a working channel of an endoscope 13. The pusher member 52 is butted up against the treatment member 12 in the working channel of the endoscope.

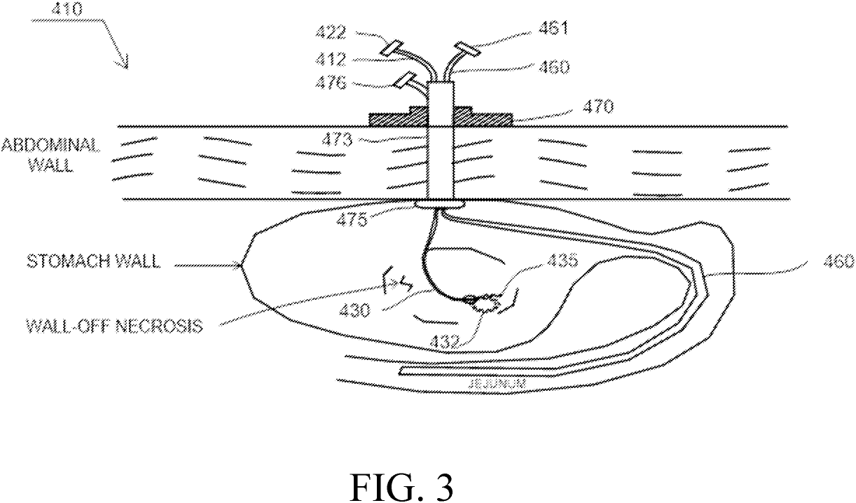

FIG. 3 shows an embodiment of a double lumen gastrointestinal catheter 410 placed within a patient by percutaneous access, a first treatment member 412 of the catheter having treatment site flushing capability, and a second treatment member 460 of the catheter having small bowel site enteral feeding capability or stomach-in fluid aspiration capability. In an embodiment, the double lumen gastrointestinal catheter 410 may include a first treatment member 412 and a second treatment member 460, the first treatment member 412 and the second treatment member 460 may be advanced into the patient through a percutaneous endoscopic gastrostomy (PEG) tube 473, and a balloon anchor 475 and an anchoring bolster 470 may be configured to stabilize the double lumen gastrointestinal catheter 410 at the percutaneous access site. The first treatment member 412 may include a joint 422, a distal potion 430, a distal end region 432 of the distal potion 430, and a distal tip 435, and the second treatment member 460 may include a joint 461. The double lumen gastrointestinal catheter 410 may further include a balloon inflation valve 476 to provide an interface for inflation of the balloon anchor 475.

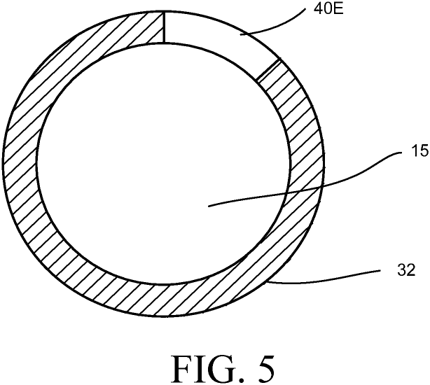

FIGS. 4A-4E show various fluid port 40 embodiments that may be disposed in a distal end region of a gastrointestinal catheter configured for flushing a treatment site, as shown in FIG. 1, and variously in FIGS. 5, 6A-6B, and 7A-7B. FIG. 4A has fluid ports configured as holes 40A, FIG. 4B has fluid ports configured as slits 40B, FIG. 4C has fluid ports configured as skives 40C, FIG. 4D has fluid ports configured as spray jets or drip sites 40D, and FIG. 4E has a fluid port configured as a single large hole 40E. These variously configured fluid port embodiments may be included within any embodiment of the disclosure.

Fluid port embodiments 40A-40E are of various dimensions, according to their configuration, but conform generally to the requirement that they are large enough to allow free flow or fluid, but not so large as to compromise the stiffness of the loop structure. As shown in FIG. 5, any particular port 40A-40E has a widest diameter no more than about 20% the diameter of the distal end region of the distal portion of any gastrointestinal catheter embodiment.

Fluid port embodiments 40A-40E are arranged such that their perpendicular axes may be disposed at multiple angles with respect to the axis of the main catheter lumen to ensure adequate flow with a wide spread pattern to minimize occlusion due to inadvertent contact with a tissue surface. The fluid flow rate through fluid port embodiments 40A-40E may vary according to a flow rate of irrigant being pushed through the catheter, the number of fluid ports, and the particularities of size and shape of the fluid ports. Fluid port embodiments 40A-40E may be distributed at substantially consistent intervals throughout the length of a distal loop, but in some embodiments, distribution intervals may be of variable length, and in the example of a multi-loop configuration, fluid ports may be disproportionately distributed among the individual loops. In one example, one distal loop of a multi-loop embodiment may be substantially or relatively free of fluid ports, the overall stiffness of the loop thus being uncompromised by fluid ports, the loop thereby having a greater stability in the preferred configuration and having greater anchoring capability.

FIG. 5 shows a cross sectional view of a large fluid port 40E within the distal portion of a gastrointestinal catheter 10 configured for flushing a treatment site. In an embodiment, the irrigant may flow through a lumen 15 of the distal end region 32 of the distal portion, and enter into a patient via the fluid port 40E. This fluid port embodiment may be included within any embodiment of the disclosure.

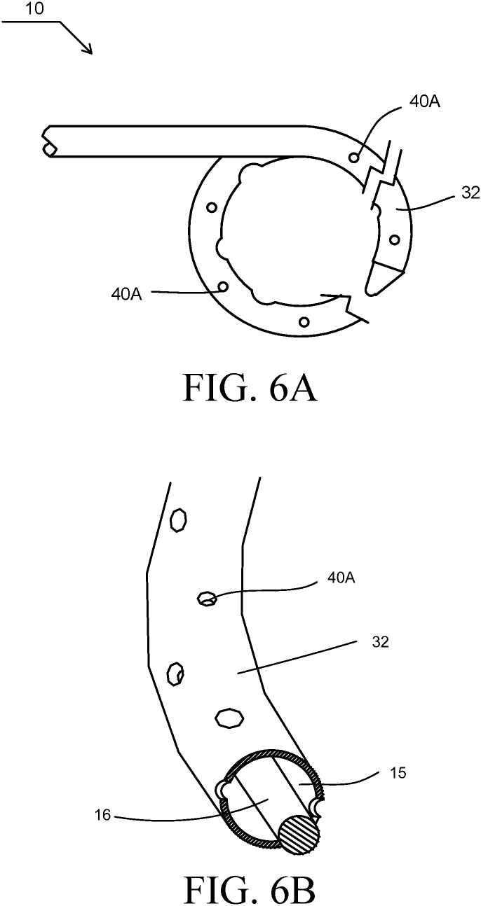

FIGS. 6A -6B show views of fluid ports 40A within the distal end region loop 32 of a gastrointestinal catheter embodiment 10 configured for flushing a treatment site. FIG. 6A shows a side view of the distal portion of a catheter that has a single loop. FIG. 6B shows a cross sectional perspective view of the distal end loop, the lumen of the catheter hosting a guidewire 16. These configurations (FIGS. 6A-6B) of a distal end region may be included in any embodiment of the disclosure.

FIGS. 7A-7C show various views of the distal end of an embodiment of a gastrointestinal catheter configured for flushing a treatment site, this particular embodiment having a distal anchoring end configured as a double loop 33b as its preferred configuration. This configuration of a distal end region may be included in any embodiment of the disclosure. FIG. 7A shows a side view, FIG. 7B shows a top view, and FIG. 7C shows a face end view. This distal end configuration may be included in any embodiment of the disclosure.

As noted elsewhere, embodiments of distal anchoring loops 32 and 33 are relatively stiff by virtue of a stiff material or geometry that resists deformation and straightening, enabling it to resist pullout from the target wound. Anchoring loop embodiments commonly have an outer diameter OD of about 25 mm (but can range from 13-50 mm OD). In the instance of a double-loop or multi-loop embodiment 33, the loops are typically about the same size, but diameters of individual loops within a plurality of loops may vary from each other.

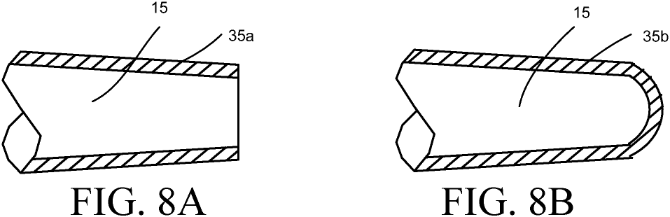

FIGS. 8A-8B show of embodiments of a distal tip 35 for the distal end of a gastrointestinal catheter configured for flushing a treatment site. FIG. 8A shows a tapered tip 35A with a distal orifice; FIG. 8B shows a bullet-shaped tip 35B with a closed end. Either of these distal tip 35 configurations may be included in any embodiment of the disclosure. Each of these embodiments has a particular advantage. Distal tip 35A (with a distal tip orifice) is particularly adapted to deliver a forward directed stream of irrigating fluid. Distal tip 35B (with a closed distal end) is particularly adapted to stop forward movement of a guidewire past the tip in embodiments of methods that use a guidewire. Stopping forward movement prevents tissue injury that could otherwise occur by an advancing guidewire.

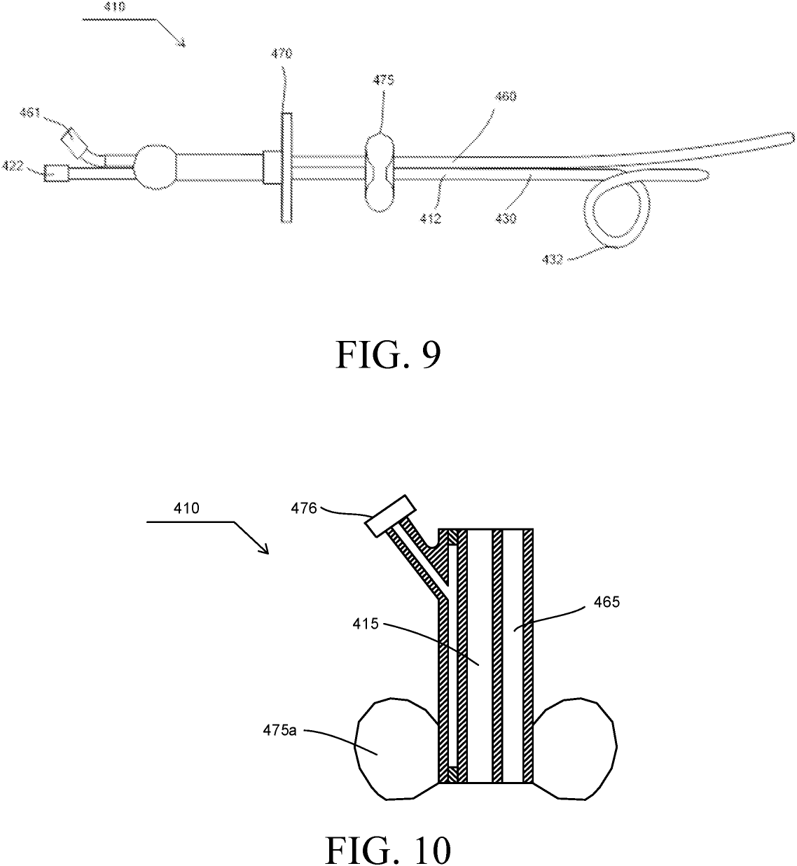

FIG. 9 shows a side view of an embodiment of double lumen gastrointestinal catheter 410 configured for access by the percutaneous endoscopic gastrostomy (PEG) tube, the catheter including a first treatment member 412 for flushing a treatment site and a second treatment member 460 for feeding into the small bowel or for aspirating fluid in the stomach.

FIG. 10 shows a view of an embodiment of a double lumen gastrointestinal catheter 410 configured for percutaneous access, the catheter having a two-balloon anchor 475a configured to stabilize the catheter at the percutaneous access site, and the double lumen gastrointestinal catheter 410 has a WON catheter lumen 415 for accommodating the first treatment member 412 and has a feeding or aspirating catheter lumen 465 for accommodating the second treatment member 460.

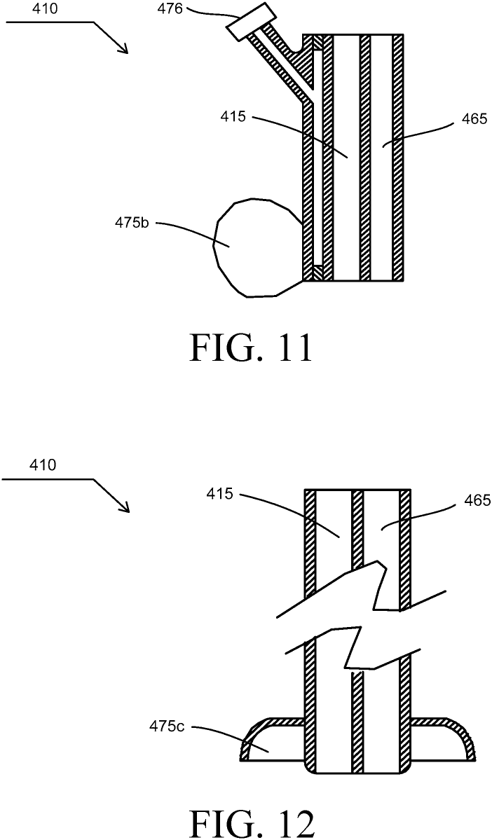

FIG. 11 shows a view of an embodiment of a double lumen gastrointestinal catheter 410 configured for percutaneous access, the catheter having an one-balloon anchor 475b configured to stabilize the catheter at the percutaneous access site.

FIG. 12 shows a view of an embodiment of a double lumen gastrointestinal catheter 410 configured for percutaneous access, the catheter having an anchor in the form of flexible skirt 475c configured to stabilize the catheter at the percutaneous access site.

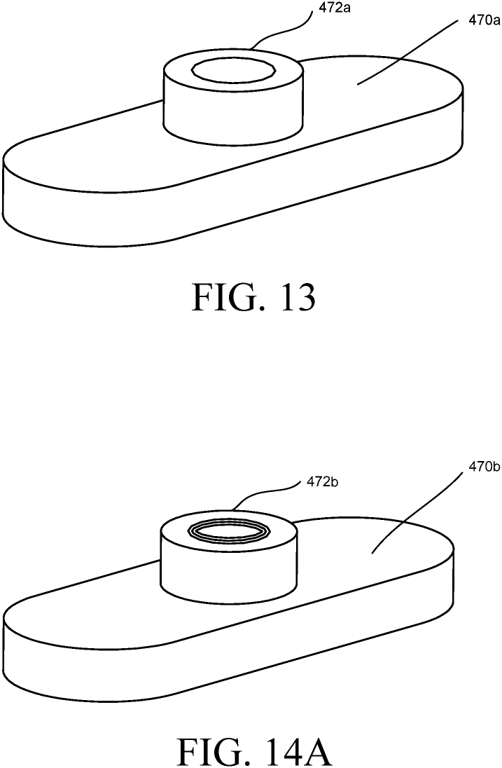

FIG. 13 shows a perspective view of an embodiment of an anchoring bolster 470a with a friction-locking feature 472a, the bolster configured to stabilize a double lumen gastrointestinal catheter 410 configured for percutaneous access.

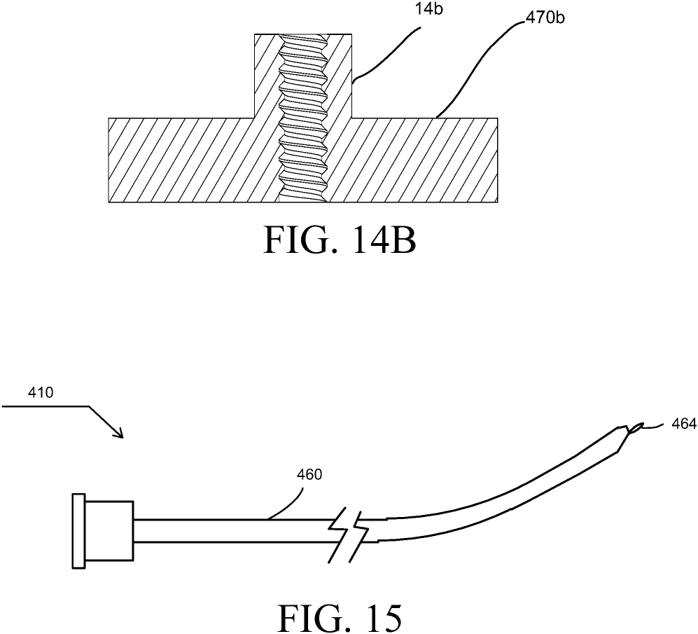

FIGS. 14A-14B show an embodiment of an anchoring bolster 470b with a locking thread feature 472b, the bolster configured to stabilize a double lumen gastrointestinal catheter configured for percutaneous access. FIG. 14A is a perspective view of a bolster 470b with a threaded connection feature 472b; FIG. 14B is a cross sectional view of threaded feature 472b of the threaded bolster 470b.

FIG. 15 shows a side view of an embodiment of a second treatment member 460 of a double lumen gastrointestinal catheter 410 configured for percutaneous access, the second treatment member 460 configured for enteral feeding into the small bowel or for aspirating fluid in the stomach. In an embodiment, the distal end of the second treatment member 460 connects with a manipulation loop 464, and after the second treatment member 460 is advanced into the patient, the manipulation loop 464 may be grasped by other device, for example, a clamp, to place the second treatment member 460 at a treatment site through an endoscope.

FIG. 16 shows an embodiment of a transitional region 25 of a gastrointestinal catheter member configured for flushing a treatment site where proximal 30 and distal 20 portions meet, and wherein the stiffness of the distal portion is greater than that of the proximal portion, the variable stiffness provided by thickness variation feature 20a of the sidewall. This embodiment of a transitional region and the featured difference in stiffness between distal and proximal portions of gastrointestinal catheter configured to flush a treatment site may be included in any embodiment of the disclosure.

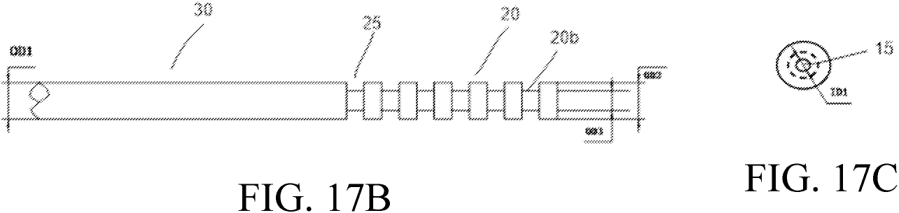

FIGS. 17A-17C show views of a transitional region 25 of a gastrointestinal catheter member configured for flushing a treatment site where proximal 30 and distal 20 portions meet, and wherein the stiffness of the distal portion is greater than that of the proximal portion, the variable stiffness provided by ridging features 20b (orthogonally aligned with respect to the main catheter axis) within the sidewall. FIG. 17A shows a perspective view, FIG. 17B shows a side view, and FIG. 17C shows a cross sectional view. This embodiment of a transitional region and the featured difference in stiffness between distal and proximal portions of gastrointestinal catheter configured to flush a treatment site may be included in any embodiment of the disclosure.

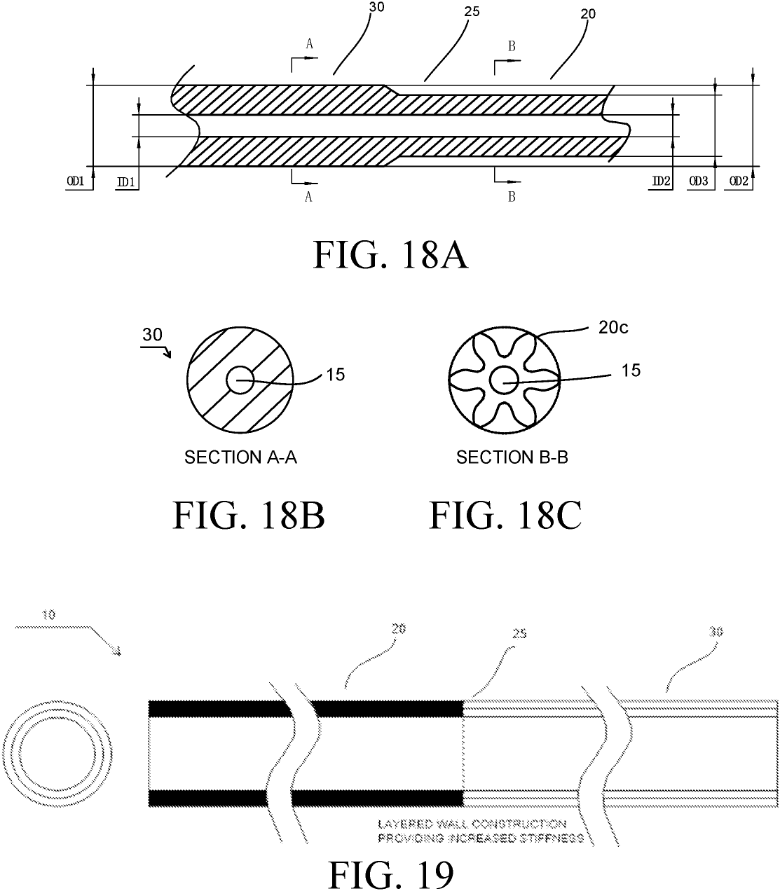

FIGS. 18A-18C show views of a transitional region 25 of a gastrointestinal catheter member configured for flushing a treatment site where proximal 30 and distal 20 portions meet, and wherein the stiffness of the distal portion is greater than that of the proximal portion, the variable stiffness provided by sidewall axially-arranged convolutional features 20c. FIG. 18A shows a side view of the catheter, FIG. 18B shows a cross sectional view of a proximal portion of the catheter, and FIG. 18C shows a cross sectional view of a distal portion of the catheter. This embodiment of a transitional region and the featured difference in stiffness between distal and proximal portions of gastrointestinal catheter configured to flush a treatment site may be included in any embodiment of the disclosure.

FIG. 19 shows views of an intersectional region of a gastrointestinal catheter where proximal and distal portions meet, and wherein the stiffness of the distal portion is greater than that of the proximal portion, the variable stiffness provided by differences in layered construction of the sidewall.

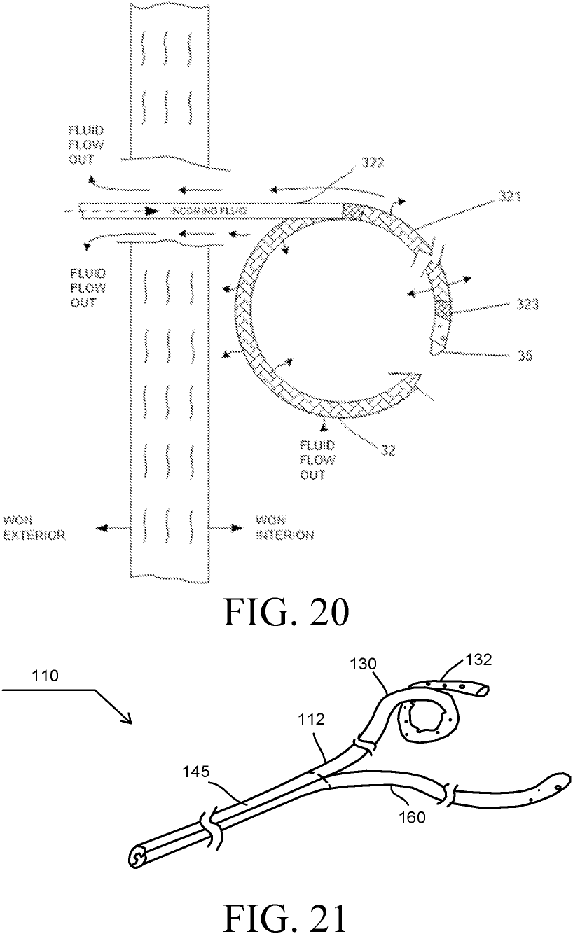

FIG. 20 shows a flow diagram for irrigation fluid (arrows) through and out a distal end region 32 of gastrointestinal catheter treatment member 12, the distal end disposed within a treatment site in need of flushing. This particular example of a distal end region comprises a nitinol segment 321. Nitinol is a shape memory metal alloy composition that assumes a preferred configuration (in this example, an anchoring configuration) but can assume a non-preferred configuration when constrained into such configuration, and can regain its preferred configuration when the segment is removed from constraint. In an embodiment, the nitinol segment 321 of the distal end region 32 of the gastrointestinal catheter treatment member 12 may be an expanded laser-cut nitinol tubing, two ends of the nitinol segment 321 may be connected with a nitinol-plastic tubing transition segment 323, and the nitinol-plastic tubing transition segment 323 may be connected with a plastic section 322.

FIG. 21 shows a side view of an embodiment of a double lumen gastrointestinal catheter 110, the catheter having a first treatment member 112 with a treatment site irrigation capability and a second treatment member 160 with a small bowel feeding capability or stomach-in fluid aspiration capability, the first treatment member 112 may include a distal portion 130 and a distal end region 132 of the distal portion 130, where the first treatment member 112 has a first lumen and the second treatment member 160 has a second lumen, and proximal portions 145 of the first treatment member 112 and the second treatment member 160 are detachably or slidably conjoinable side by side. This embodiment of a double lumen catheter may be proximally configured for either nasal access or abdominal percutaneous access.

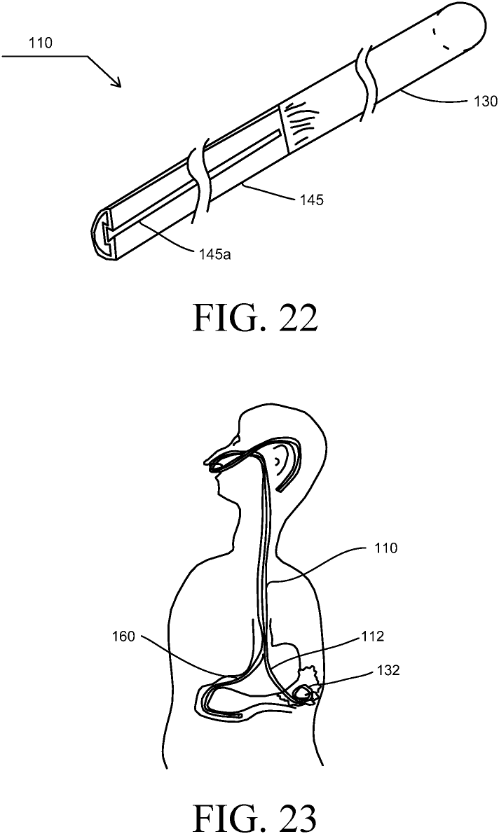

FIG. 22 shows one of the treatment members of an embodiment of a double lumen gastrointestinal catheter 110 having an axially-aligned dovetail joint 145a that connects the two lumens in a conjoined proximal portion 145 of the catheter, the distal portions of the two members being separate distal to conjoined section 145, the lumen of each member being hemi-circular in the proximal conjoined region and fully circular in the distal end region. This example of a double lumen catheter may be proximally configured for as an embodiment either nasal access or abdominal percutaneous access.

FIG. 23 shows an embodiment of a double lumen gastrointestinal catheter 110 as placed within a patient, catheter 110 having a first treatment member 112 configured for treatment site flushing capability and a second member 160 configured for small bowel enteral feeding capability or stomach-in fluid aspiration capability.

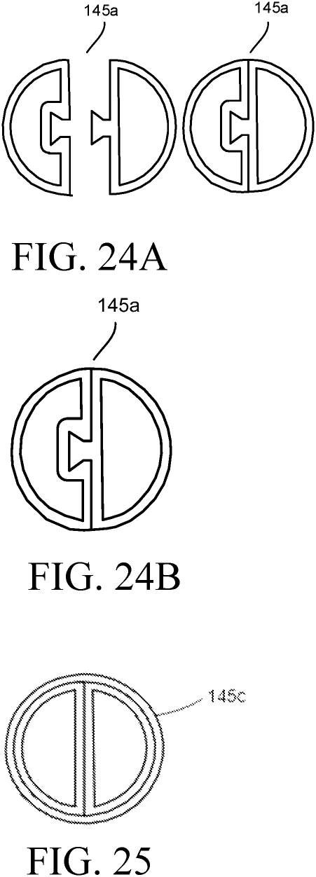

FIGS. 24A-24B show cross-sectional views of dovetail joint features 145a in a conjoined section 145 of an embodiment of a double lumen gastrointestinal catheter. FIG. 24A shows the two luminal portions disengaged; FIG. 24B shows the two luminal portions joined together by the dovetail joint. FIG. 25 shows a cross-sectional view of conjoined section 145 as an embodiment of a double lumen gastrointestinal catheter, the two lumens conjoined by an outer layer sheath 145c. Embodiments of a double lumen gastrointestinal catheter 110, as shown in FIG. 24A-25, when conjoined, side-be-side, generally form a circular diameter, each of the two lumens (corresponding to first and second treatment members) have a D-shaped cross-sectional profile.

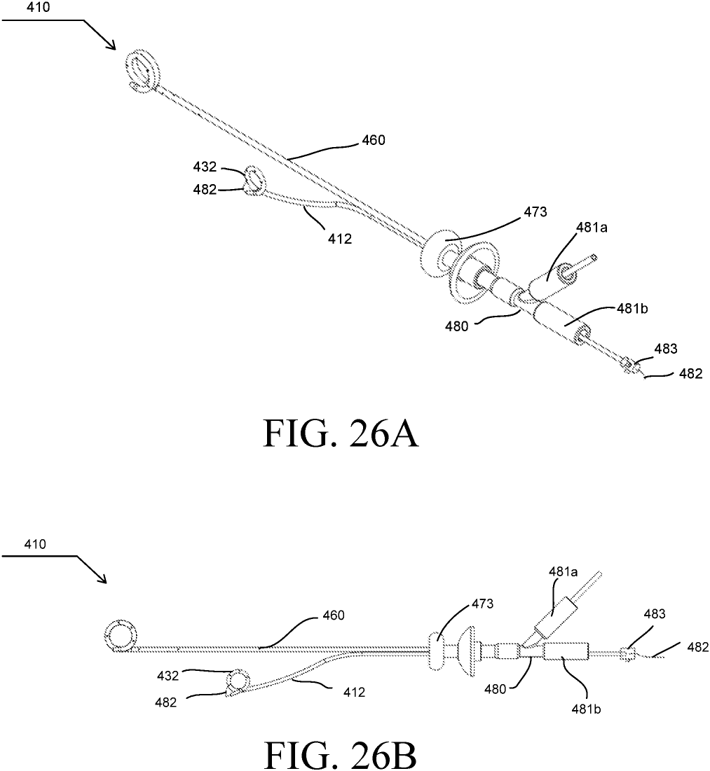

FIG. 26A shows a perspective view of an embodiment of a double lumen gastrointestinal catheter 410 for percutaneous access, FIG. 26B shows a side view of an embodiment of a double lumen gastrointestinal catheter 410 for percutaneous access, the double lumen gastrointestinal catheter 410 includes a connection member 480 and sealing caps 481. The connection member 480 may have three connection ends, where one connection end is connected to an end of the percutaneous endoscopic gastrostomy (PEG) tube 473 located close to the outside of a patient’s body, and two other connection ends are a first connection end and a second connection end, respectively. The first connection end and the second connection end are connected with the first sealing cap 481a and the second sealing cap 481b, respectively. The first sealing cap 481a and the second sealing cap 481b are each internally provided with a sealing part, so that when the first sealing cap 481a is connected with the first connection end and also when the second sealing cap 481b is connected with the second connection end, sealing is formed. The sealing can prevent the liquid in a patient from flowing out, and prevent the first treatment member 412 and the second treatment member 460 from moving axially after the first treatment member 412 and the second treatment member 460 pass through the first sealing cap 481a and the second sealing cap 481b and reach the first treatment site and the second treatment site, respectively. The sealing part may be a sealing gasket.

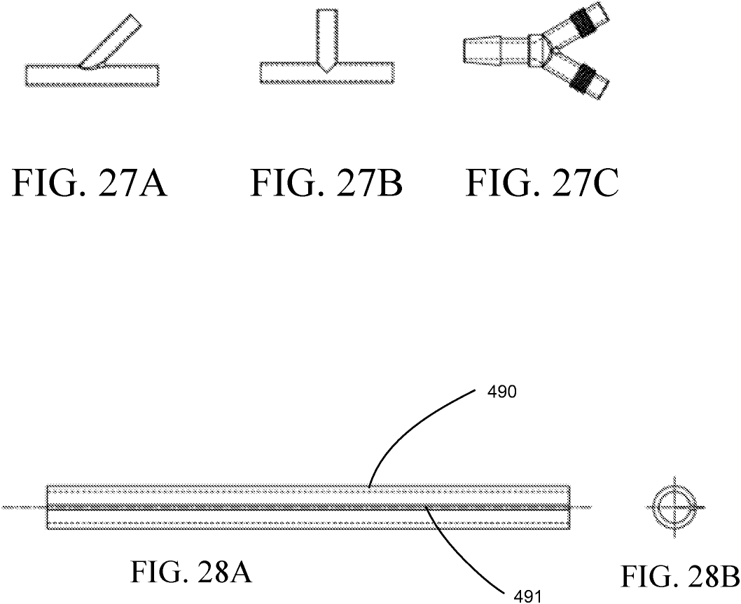

The first treatment member 412 is provided with a traction wire 482, and before the first treatment member 412 enters into the patient’s body through the connection member 480 and is fixed and sealed at the first treatment site of the patient’s body, the traction wire 482 can be pulled in a direction facing outside of the patient, so that the distal end region 432 of the distal portion of the first treatment member 416 (i.e. the region on the side where the first treatment site is located) assumes a loop-like configuration. The loop-like configuration is used to fix the distal end region 432 of the distal portion of the first treatment member 412 to the first treatment site, so as to prevent the distal end region 432 of the distal portion of the first treatment member 412 from detaching from the first treatment site. A third sealing cap 483 is arranged at the end of the proximal portion of the first treatment member 412 to prevent the liquid of the first treatment member 412 from flowing out of the body through a first lumen of the first treatment member 412. The connection member 480 may be T-shaped, Y-shaped, etc., and the present disclosure does not define forms thereof. FIGS. 27A-27C show three forms of the connection member 480. The first connection end and the second connection end of the connection member 480 can be connected with the first sealing cap 481a and the second sealing cap 481b respectively by, for example, a thread, a buckle, etc. in a detachable manner. The present disclosure does not limit the connection method, as long as the connection and sealing can be realized.

After the first treatment member 412 enters the percutaneous endoscopic gastrostomy (PEG) tube 473 through any one of the first connection end and the second connection end of the connection member 480, the second treatment member 460 can enter the percutaneous endoscopic gastrostomy (PEG) tube 473 through the other connection end of the first connection end and the second connection end of the connection member 480; or after the second treatment member 460 may enter the percutaneous endoscopic gastrostomy (PEG) tube 473 through any one of the first connection end and the second connection end of the connection member 480, the first treatment member 412 may enter the percutaneous endoscopic gastrostomy (PEG) tube 473 through the other connection end of the first connection end and the second connection end of the connection member 480, so that the first treatment member 412 and the second treatment member 460 may be put into or pulled out of the patient’ body metachronously.

The gastrointestinal catheter 410 further includes a straight rectifying tube 490, which is a straight hollow tube and has openings at both ends. The straight rectifying tube 490 has a length enough to straighten the loop-like configuration of the distal end region 432 of the distal portion of the first treatment member 412 (i.e. the region on the side where the first treatment site is located), usually has a length of 50-200 mm, and a diameter of the straight rectifying tube 490 is adapted to or slightly larger than the diameter of the distal end region 432 of the distal portion of the first treatment member 412, so that the distal end region 432 of the distal portion of the first treatment member 412 may pass inside the straight rectifying tube 490. FIG. 28A show a side view of the straight rectifying tube 490 with a seam 491 on its side wall, and FIG. 28B shows the cross-sectional view of the straight rectifying tube 490. Before advancing the first treatment member 412 to the first treatment site, the straightening of the distal end region 432 of the distal portion of the first treatment member 412 is carried out by passing the distal end region 432 of the distal portion of the first treatment member 432 through the straight rectifying tube 490 , and then the first treatment member 412 is advanced into the patient’s body distally along a guidewire, wherein the straightening of the distal end region 432 of the distal portion 430 of the first treatment member 412 specifically includes advancing the straight rectifying tube 490 independently, and when the straight rectifying tube 490 entirely passes the first treatment member 412, is moved to and around the guidewire, and finally entirely passes the guidewire, taking out the straight rectifying tube 490 along the seam 491 of the straight rectifying tube 490.

The distal end region 432 of the distal portion 430 the first treatment member 412 is in a linear constrained configuration; after the guidewire is pulled out, the distal end region 432 of the distal portion of the first treatment member 412 recovers to the substantial loop configuration, which may be formed by pulling the traction wire 482 disposed on the first treatment member 412.

In some embodiments, the second treatment member is configured to provide an enteral feeding solution into the small bowel of a patient or is placed into the stomach to aspirate fluid. This would allow for a closed-circuit mechanism whereby fluid is instilled into the walled-off necrosis (WON) through the first treatment member and then aspirated (or suctioned out) through the second treatment member (a capability/ability of aspirating such fluid through the second treatment member is called as stomach-in fluid aspiration capability/functionality in the context of the present disclosure). This would be desirable in a patient with, for example, gastroparesis to prevent reflux of fluid contents collecting in the stomach. The ability to aspirate through the second treatment member may also be of benefit solely for decompression of the stomach on an ‘as needed’ basis (can be activated when needed or desired). As a specific example, aspiration would be desirable when hydrogen peroxide is instilled into the WON since this bubbles up and expands, which may result in reflux and aspiration of hydrogen peroxide if not suctioned out in a closed-circuit manner.

Four basic embodiments of the disclosure are provided as devices; embodiments of the disclosure further include methods of placing and operating embodiments of these devices. In the description that follows, each embodiment as depicted in FIGS. 1-25, will be described.

A first embodiment of the disclosure includes a gastrointestinal catheter sized and configured for access into the gastrointestinal tract, the catheter including a treatment member configured to treat an anatomical site in medical need of flushing with an irrigating fluid. Some embodiments of the first embodiment include a single lumen and are sized and configured for a nasal pathway into the gastrointestinal tract.

A second embodiment of the disclosure includes a double-lumen gastrointestinal catheter sized and configured for nasal entry into the gastrointestinal tract, the catheter including a first and a second treatment member, where the first treatment member has a first lumen and the second treatment member has a second lumen, and the proximal portions of the first treatment member and the second treatment member are detachably and slidably conjoinable side by side. The first treatment member is configured to treat an anatomical site in medical need of flushing with an irrigating fluid; in some embodiments, the second treatment member is configured to provide an enteral feeding solution into the small bowel of a patient or aspirate fluid in the stomach of a patient. There are advantages to the dual functionality of this embodiment; it is common for a patient in need of having a necrotic site flushed (as, for example, by a first treatment member) to also have a need for enteral feeding (as, for example, by a second treatment member), or for a patient in need of flushing a necrotic site (as, for example, by a first treatment member) and meanwhile having a need for aspirating fluid in the stomach of a patient (as, for example, by a second treatment member). This embodiment, thus, advantageously and efficiently provides for both capabilities in a single device.

A third embodiment of the disclosure includes a gastrointestinal catheter sized and configured for abdominal percutaneous access into the gastrointestinal tract, the catheter including a treatment member configured to treat an anatomical site in medical need of flushing with an irrigating fluid.

A fourth embodiment of the disclosure includes a double-lumen gastrointestinal catheter sized and configured for abdominal percutaneous access into the gastrointestinal tract, the catheter including a first and a second treatment member, where the first treatment member has a first lumen and the second treatment member has a second lumen, and the proximal portions of the first treatment member and the second treatment member are detachably or slidably conjoinable side by side. The first treatment member is configured to treat an anatomical site in medical need of flushing with an irrigating fluid; the second treatment member is configured to provide an enteral feeding solution into the small bowel of a patient or aspirate fluid in the stomach of a patient. There are advantages to the dual functionality of this fourth embodiment; it is common for a patient in need of having a necrotic site flushed (as, for example, by a first treatment member) to also have a need for enteral feeding (as, for example, by a second treatment member), or for a patient in need of flushing a necrotic site flushed (as, for example, by a first treatment member) and meanwhile having a need for aspirating fluid in the stomach of a patient (as, for example, by a second treatment member). This fourth embodiment, thus, advantageously and efficiently provides for both capabilities in a single device, which requires only one site of entry into the gastrointestinal tract by way of an abdominal percutaneous access site.

### Embodiment 1 Device: A Gastrointestinal Catheter (Nasal Entry, Single Treatment Member)

In a first embodiment of the disclosure, a gastrointestinal catheter configured for treatment of a site in medical need of treatment by flushing, irrigation, or drainage is provided. This first embodiment of a gastrointestinal catheter includes a lumen, is sized and configured to enter the body by way of a nasal entry route and to reach a treatment site in the gastrointestinal tract. One example of a site in need of such treatment is the pancreas, where acute or chronic pancreatitis can result in infected or necrotic encapsulated pockets that are commonly referred to as walled-off pancreatic necrosis. Aspects of the first embodiment of the disclosure are described further below in the context of the description of FIGS. 1, 2, 4-8, and 16-19.

Accordingly, a gastrointestinal catheter for insertion into a treatment site in a patient includes a treatment member sized to fit within an endoscope, the treatment member including a lumen, a proximal portion, a distal portion, and a distal end region within the distal portion. The distal end region of the treatment member has a preferred configuration, but the treatment member is capable of elastically assuming a substantially linear constrained configuration, as may occur when, for example, a guidewire is disposed within the lumen, or when the treatment member is being hosted within the working channel of an endoscope. The preferred configuration is adapted to anchor the distal end region within the treatment site, and absent constraint, will assume its preferred and anchoring configuration. The distal portion of the treatment member has a higher level of stiffness than that of the proximal portion of the treatment member. The distal end region of the treatment member includes one or more fluid ports, in communication with the lumen, that are configured to allow through flow of a fluid.

Stiffness may be characterized in terms of the modulus of elasticity; the distal portion of the treatment member of embodiments of gastrointestinal catheter has a higher modulus of elasticity than that of the proximal portion. Accordingly, in some embodiments, the distal portion of the treatment member has an elastic modulus in the range of about 40,000 psi to about 600,000 psi. In some embodiments, the distal portion of the treatment member has an elastic modulus in the range of about 40,000 psi to about 300,000 psi. In some embodiments, the distal portion of the treatment member has an elastic modulus in the range of about 40,000 psi to about 150,000 psi. And in some embodiments, the distal portion of the treatment member has an elastic modulus in the range of about 40,000 psi to about 75,000 psi. By way of example, a product of Arkema provides a polyether block amide product, PEBAX® 6333, having an elastic modulus of 41,300 psi.

The elastic modulus ranges of various embodiments of the distal portion of the gastrointestinal catheter do not necessarily exclude embodiments of the proximal portion from those ranges so long as the elastic modulus of the proximal portion is less than that of the distal portion. Accordingly, when the distal portion of the treatment member has an elastic modulus greater than about 75,000 psi, the proximal portion of the treatment member can have an elastic modulus in the range of about 1,000 psi to about 75,000 psi. When the distal portion of the treatment member is greater than about 42,000 psi, the proximal portion of the treatment member can have an elastic modulus in the range of about 1,500 psi to about 42,000 psi.

In a functional sense, in some embodiments of the gastrointestinal catheter, the distal portion of the treatment member has a sufficiently high elasticity that upon insertion into the treatment site and release from a substantially linear constrained configuration, the distal end region assumes the preferred and anchoring configuration.

In some embodiments, the proximal portion of the treatment member comprises an elastic modulus in the range of about 1,700 psi to about 25,000 psi. By way of example, a product of Arkema provides a polyether block amide product, PEBAX® 2533, having an elastic modulus of 1,750 psi and PEBAX® 633 having an elastic modulus of 41,300 psi.

In a functional sense, in some embodiments of the gastrointestinal catheter, the proximal portion of the treatment member comprises an elastic modulus that provides a level of compliance that mitigates a possibility of injury to the patient in a facial area proximate a nasal site of entry of the treatment member.

In some embodiments, the distal end region of the treatment catheter is substantially equivalent in length to the distal portion of the treatment catheter. In other embodiments, the distal end region of the treatment catheter is less in length than the total length distal portion of the treatment catheter.

Various physical and structural properties of the treatment member of the gastrointestinal catheter can underlie the difference in elastic modulus between the proximal and distal portions of the treatment member. Accordingly, in some embodiments, the elasticity of the distal end region of the treatment member is higher than that of the proximal portion by virtue of a difference in a material composition.

In some embodiments, the elasticity of the distal portion of the treatment member is higher than that of the proximal portion by virtue of a difference in a sidewall structure of the treatment member. For example, in some embodiments, the difference in a sidewall structure of the treatment member includes a difference in thickness of the sidewall, as when the distal portion of the treatment member includes a region of greater sidewall thickness than that of the proximal portion of the treatment member. In some embodiments, the difference in a sidewall structure of the treatment member includes a plurality of indents or inward axial convolutions in the sidewall of the proximal portion of the treatment member, wherein an outer diameter of the indents or inward convolutions is less than an outer diameter of the distal portion of the treatment member. In still further embodiments, the difference in a sidewall structure of the treatment member includes variation in one or more layers of varying elastic modulus in the sidewall.

A treatment site for which embodiments of a gastrointestinal catheter is medically appropriate is typically an anatomical cavity, often the anatomical cavity is inflamed, infected, necrotizing, or otherwise abnormal or pathological. One particular example of such an anatomical cavity is an encapsulated or walled-off necrotic site involving the pancreas, wherein the distal end region of the treatment member is sized and configured for insertion into such an encapsulated pancreatic necrotic site.

Some embodiments of a gastrointestinal catheter include a guidewire hosted within the lumen of the treatment member. In a functional sense, this embodiment typically occurs as the treatment member is passed over the guidewire, as the treatment member is being advanced toward a treatment site. In particular embodiments, the guidewire is sufficiently stiff to maintain the distal end of the treatment member (in which it is disposed) in a substantially linear constrained configuration.

As noted above, in some embodiments, the preferred configuration of the distal end region of the treatment member is one that anchors that region in the treatment site, and accordingly, anchors the treatment member, as a whole. Merely by way of example, an anchoring configuration may take the form of a loop or multiple loops.

The functional purpose of the preferred configuration of the distal end region of the treatment member is to anchor it in a treatment site, such as, merely by way of example, a walled off necrotic site involving the pancreas. By the distal end region anchoring in the treatment site, the gastrointestinal catheter, as a whole, is anchored in the treatment site. By way of exemplifying an anchoring configuration, the preferred configuration of the distal end region of the distal portion of the treatment may be configured as a loop. In some embodiments, the preferred configuration includes two or more loops.

It is also advantageous that the distal end of the treatment member not injure the treatment site; accordingly, in some embodiments the distal end region of the treatment member includes an atraumatic tip. In some embodiments, the distal end region of the treatment member also includes a distal end fluid port in communication with the lumen.

Another aspect of the distal end region of embodiments of the gastrointestinal catheter relates to the fluid ports referred to above. In some embodiments, the fluid ports are positioned within the distal end region of the first treatment member such that when the distal end is anchored within the treatment site, the fluid ports are disposed in their substantial entirety within the treatment site.

Various aspects of the distal end region of the treatment member of the first embodiment of a gastrointestinal catheter relate to that region being formed, at least in part, by a shape memory composition. Accordingly, some embodiments of the gastrointestinal catheter have a higher level of stiffness in the distal portion of the treatment member (compared to the proximal portion) that is at least partly attributable to a segment disposed within the distal portion having a shape memory composition. Some embodiments of the distal end region of the treatment member have a segment that includes a shape memory composition, such as a shape memory polymer or a shape memory metal alloy. In some embodiments, the segment having a shape memory composition has a preferred configuration that is adapted to anchor the distal end of the treatment member within the treatment site.

By way of example, the shape memory composition may include nitinol in any appropriate form or configuration, such as wires in the form of any of a braid or axially-aligned linear wires. In some embodiments of the gastrointestinal catheter, the distal end region consists in its substantial entirety of nitinol. In some embodiments, the shape memory alloy segment is disposed within the lumen of the treatment member, against an inner surface of a treatment member sidewall. In some embodiments, the shape memory alloy segment is disposed within a sidewall of the treatment member, sandwiched between an inner and an outer polymer layer.

The dimensions of embodiments of the treatment member of a gastrointestinal catheter be appropriate both for conveyance through an endoscope and appropriate for the anatomical dimensions of the routing to a treatment site. Accordingly, in some embodiments of the gastrointestinal catheter, the outer diameter of the treatment member ranges from about 3 French to about 13 French. In some embodiments, the outer diameter of the treatment member ranges from about 5 French to about 11 French. In typical embodiments, the length of the treatment member is appropriate and sufficient to reach the treatment site by way of a route that originates in the nostril of the patient.

Some embodiments of the disclosure include a gastrointestinal catheter and other ancillary items to form of a complete kit. Accordingly, such a kit includes a gastrointestinal catheter is described above, a pusher member sized and configured to fit within the endoscope, the pusher member having a lumen. In some embodiments, the kit may further include a guidewire sized and configured to be hosted within the lumens of both the pusher member and the treatment member. The guidewire has a distal tip, and the guidewire is of sufficient length that its distal tip can extend beyond the distal end region of the treatment member.

### Embodiment 1 Method: Flushing a Treatment Site With a Nasal Entry Catheter

Embodiments of the disclosure also include a method of flushing a treatment site with an embodiment of a gastrointestinal catheter as described above, by way of the treatment member. Accordingly, the method includes distally advancing an endoscope from an oral entry site to a position wherein a distal end of the endoscope is positioned proximate or within an entry route into the treatment site, the treatment member hosted in a working channel of the endoscope. The method further includes advancing the treatment member distally from the endoscope, a distal end of the treatment member thereby entering the treatment site, and then anchoring the distal end region of the treatment member within the treatment site. The method then further includes withdrawing the endoscope from the patient, and then flowing an irrigation fluid through the treatment member and into the treatment site.

In typical embodiments of the method, as described elsewhere, following the withdrawal of the endoscope, the proximal portion of the treatment member is rerouted from its oral entry site to the nasal passage, where it remains during a course of medical treatment.

Some embodiments of the method of flushing a treatment site make use of a treatment member in which the distal end region of the treatment member has a preferred anchoring configuration but is also capable of elastically assuming a substantially linear constrained configuration. In such embodiments of the method, anchoring the distal end region of the treatment member within the treatment site may include releasing the distal end region from confinement within the endoscope, thereby allowing the distal end region to reconfigure into its preferred anchoring configuration.

Some embodiments of the method of flushing a treatment site make use of a treatment member in which the distal end region of the treatment member includes a segment having a shape memory composition. In such embodiments of the method, allowing the distal end region to reconfigure into its preferred anchoring configuration includes the shape memory segment reconfiguring from the constrained linear configuration to the preferred configuration.

Some embodiments of the method of flushing a treatment site make use of a pusher member to assist in the placement of a treatment member in a treatment site. Accordingly, in some embodiments of the method, withdrawing the endoscope from the patient comprises simultaneously (a) withdrawing the endoscope from the patient while (b) distally advancing a pusher member within the endoscope so as to stabilize the anchored position of the distal end region of the treatment member within the treatment site. This type of maneuver may be referred to as a “one-to-one scope exchange” technique by physicians practiced in the art. In some embodiments of the method, a stylet or guidewire is inserted into the treatment member and advanced until its distal end is within the distal end region of the treatment member. The advancement of the straight stylet or guidewire within the distal end region straightens the end region from its preferred and anchoring configuration, and allows for an easy withdrawal of the distal end region from the anatomical site in which it had been anchored.

Some embodiments of the method of flushing a treatment site make use of guidewire to facilitate placement of a treatment member in a treatment site. Accordingly, in some embodiments of the method, prior to distally advancing the endoscope from the oral entry site, the method includes advancing a guidewire from the oral entry site until a distal tip of the guidewire is situated within the treatment site. In some embodiments, distally advancing the endoscope may include advancing the treatment member within the endoscope over the guidewire until the distal end of the treatment member is within the treatment site. Some embodiments of the method further include withdrawing the guidewire from treatment site, leaving the distal end of the treatment member there within, the distal end of the treatment member thereby being allowed to assume its preferred configuration and anchoring within the treatment site.

In practicing an embodiment of the method of flushing a treatment site, entry of the distal end of the treatment member into the treatment site may be difficult. In such an incident, the method (prior to distally advancing the endoscope to a position such that the distal end of the endoscope is positioned proximate an entry route into the treatment site) may include placing a stent within the entry route into the treatment site, and thus advancing the treatment member distally from the endoscope may include advancing the treatment member through an opening provided by the stent.

Some embodiments of the method of flushing a treatment site, as above, prior to flowing the irrigation fluid through the treatment member, may include rerouting the proximal end of the treatment member from its original emergence through the patient’s mouth to an emergence through the patient’s nose or nasal passage. In particular embodiments of the method, wherein the proximal portion of the treatment member has an elastic modulus in the range of 1,000 psi to about 75,000 psi, this elastic modulus range (a) facilitates the rerouting of the treatment member and (b) mitigates a risk of patient injury during a treatment period in comparison to an ease in rerouting a treatment member and the risk of patient injury were treatment member to have an elastic modulus greater than 75,000 psi. Mitigation of risk follows from the compliance of tubing having a low elastic modulus. In general, compliant tubing is more patient-friendly than less compliant tubing.

In addition to flowing a fluid into the treatment site, as above, embodiments of the method may include removing fluid from the treatment site. Accordingly, some embodiments of the method include draining fluid from the treatment site. Draining fluid from the treatment site may further include removing cellular debris from the treatment site. Draining fluid from the treatment site may include both (a) draining an irrigation fluid that has been injected into the site as well as (b) draining a fluid from any other source within the treatment site. In particular embodiments of the method, draining fluid may include withdrawing a fluid from the treatment site through fluid ports within the distal end region of the treatment member. Draining fluid from the treatment site may be facilitated by a vacuum or suction-based draw of fluid in a distal to proximal path.

Some embodiments of the method of flushing a treatment site, as above, may include mixing an active agent into the irrigation fluid prior to the flowing the irrigation fluid into the treatment site. By way of example, an active agent may include any of an anti-biotic agent or an anti-infective agent, or any pharmaceutically effective agent. In some embodiments, the active agent may be considered to be substantially inert, pharmaceutically, but serve another medical purpose, such as may be provided by an imaging compound.

Embodiments of the method of flushing a treatment site, as above, may be directed to any gastrointestinal site in need of such treatment. In a particular example, the treatment site is a walled-off pancreatic necrotic site. In typical embodiments of the method, the treatment member remains in place, the distal end region of the treatment member within the treatment site, for a treatment period whose duration is determined by a physician. Upon completion of a medically appropriate duration, the method concludes by withdrawing the treatment member from the treatment site.

### Embodiment 2 Device and Method: A Double Lumen Gastrointestinal Catheter (Nasal Entry, Two Treatment Members)

A second embodiment of the disclosure includes a double lumen gastrointestinal catheter for insertion into a gastrointestinal tract of a patient in need of a gastrointestinal intervention at a first treatment site and a second treatment site. The embodiment is sized and configured to gastrointestinal access by way of a nasal passage entry route.

Such a double lumen gastrointestinal catheter includes a first treatment member and a second treatment member, each member having a lumen, a proximal portion, a distal portion having a distal end. The first treatment member substantially corresponds to the treatment member provided in the first embodiment of the disclosure, as described above. Aspects of the second embodiment of the disclosure are described further below in the context of the description of FIGS. 4-8, 16-19, and 20-25.

The proximal portions of the first and second treatment members are detachably or slidably conjoinable side by side, the proximal portions of the first and second treatment members are conjoined; the distal portions of the first and second treatment members are separate from each other. The first treatment member is sized and configured for insertion of its distal end into the first treatment site in an upper portion of the gastrointestinal tract; the second treatment member is sized and configured for placement of its distal end into a second treatment site in a small bowel or in the stomach of the patient. The distal end region of the first treatment member has a preferred configuration, the first treatment member is also capable of elastically assuming a substantially linear constrained configuration, wherein the preferred configuration is adapted to anchor the distal end region within the targeted treatment site. The distal portion of the first treatment member has a higher level of stiffness than that of the proximal portion of the first treatment member. The distal end region of the first treatment member includes one or more fluid ports in communication with the lumen; fluid ports are configured to allow through flow of a fluid. The fluid ports are positioned within the distal end of the first treatment member such that when the distal end is anchored within the first treatment site, the fluid ports are disposed within the first treatment site.

Embodiments of the disclosure also include a method of flushing a first treatment site and feeding a patient directly at a second treatment site with a double lumen gastrointestinal catheter that include providing a double lumen gastrointestinal catheter having a first treatment member and a second treatment member, where the first treatment member has a first lumen and the second treatment member has a second lumen, and the proximal portions of the first treatment member and the second treatment member are detachably or slidably conjoinable side by side. The method includes distally advancing the second treatment member into the second treatment site in a small bowel of the patient, followed by distally advancing the first treatment member over a guidewire to a position such that a distal end region of the first treatment member is positioned within the first treatment site. The method continues by withdrawing the guidewire proximally from the first treatment member, thereby anchoring the distal end region of the first treatment member within the first treatment site, and then withdrawing the endoscope from the patient. The method then continues by flowing an irrigation fluid through the first treatment member and into the first treatment site, and flowing a solution through the second treatment member into the second treatment site or aspirating a fluid at the second treatment site through the second treatment member.

### Embodiment 3 Device and Method: A Gastrointestinal Catheter (Percutaneous Entry, Single Treatment Member)

In a third embodiment of the disclosure, a gastrointestinal catheter for insertion into a gastrointestinal tract of a patient in need of a gastrointestinal intervention at a site that has become necrotic, infected and/or encapsulated. The embodiment is sized and configured to gastrointestinal access by way of an abdominal percutaneous entry route.

Accordingly, this third embodiment includes a treatment member that has a lumen, a proximal portion, a distal portion having a distal end region. The distal portion of the treatment member has a higher level of stiffness than that of the proximal portion of the first treatment member.

The distal end region of the treatment member of catheter includes a number of features; it has a preferred anchoring configuration that is adapted to anchor the distal end region within the first treatment site; the distal end region of first treatment member is further capable of elastically assuming a substantially linear constrained configuration. The distal end region of the treatment member has one or more fluid ports in communication with the lumen through the sidewall of the treatment member that are configured to allow through flow of a fluid.

Embodiments of the disclosure also include a method of flushing a treatment site with an embodiment of a third embodiment of the disclosure as described above, by way of a treatment member. Accordingly, a method of flushing a treatment site with a treatment member includes placing a percutaneous endoscopic gastrostomy (PEG) tube in the patient thereby providing an entry route for the treatment member. The method includes distally advancing the treatment member over a guidewire to a position such that a distal end region of the first treatment member is positioned within a treatment site, and then withdrawing the guidewire proximally from the first treatment member, thereby anchoring the distal end region of the treatment member within the first treatment site. The method then continues with withdrawing the endoscope from the patient, followed by flowing an irrigation fluid through the treatment member and into the treatment site.

### Embodiment 4 Device A Double Lumen Gastrointestinal Catheter (Percutaneous Entry Two Treatment Members, Providing Irrigation and Feeding Functionalities)

In a fourth embodiment of the disclosure, a double lumen gastrointestinal catheter for insertion into a gastrointestinal tract of a patient in need of a gastrointestinal intervention at a first treatment site and at a second treatment site is provided. Accordingly, this fourth embodiment includes a first treatment member and a second treatment member, wherein the first treatment member has a proximal portion, a distal portion having a distal end region, and a lumen through both the proximal and distal portions, where the first treatment member has a first lumen and the second treatment member has a second lumen. The proximal portions of the first and second treatment members are detachably or slidably conjoinable side by side. In one embodiment, the proximal portions of each of the first and second treatment members may be adapted to be conjoinable side-by-side, and the distal portions of the first and second treatment members may be separate from each other.

The first treatment member of the double lumen gastrointestinal catheter is sized and configured for insertion of its distal end into the first treatment site in an upper portion of the gastrointestinal tract and the second treatment member is sized and configured for placement of its distal end into a second treatment site within a bowel of the patient. In some embodiments, the distal portion of the first treatment member has a higher level of stiffness than that of the proximal portion of the first treatment member.

The distal end region of the first treatment member of double lumen gastrointestinal catheter includes a number of features; it has a preferred anchoring configuration that is adapted to anchor the distal end region within the first treatment site; the distal end region first treatment member further being capable of elastically assuming a substantially linear constrained configuration. The distal end region of the first treatment member has one or more fluid ports in communication with the lumen through the sidewall of the treatment member that are configured to allow through flow of a fluid.

Various features of double lumen gastrointestinal catheter are related to stiffness or the elastic modulus of the first treatment member closely parallel the analogous features of the treatment member of the first embodiment of a gastrointestinal catheter, as described above. As noted above, in some embodiments, the distal portion of the first treatment member has a higher level of stiffness than that of the proximal portion of the first treatment member.

Stiffness may be characterized in terms of the modulus of elasticity; the distal portion of the first treatment member of the double lumen gastrointestinal catheter has a higher modulus of elasticity than that of the proximal portion. Accordingly, in some embodiments, the distal portion of the first treatment member has an elastic modulus in the range of about 40,000 psi to about 600,000 psi. In some embodiments, the distal portion of the first treatment member has an elastic modulus in the range of about 40,000 psi to about 300,000 psi. In some embodiments, the distal portion of the first treatment member has an elastic modulus in the range of about 40,000 psi to about 150,000 psi. And in some embodiments, the distal portion of the first treatment member has an elastic modulus in the range of about 40,000 psi to about 75,000 psi. By way of example, a product of Arkema provides a polyether block amide product, PEBAX® 6333, having an elastic modulus of 41,300 psi.

The elastic modulus ranges of various embodiments of the distal portion of the double lumen gastrointestinal catheter do not necessarily exclude embodiments of the proximal portion from those ranges so long as the elastic modulus of the proximal portion is less than that of the distal portion. Accordingly, when the elastic modulus of the distal portion of the first treatment member is greater than about 75,000 psi, the proximal portion of the first treatment member can have an elastic modulus in the range of about 1,000 psi to about 75,000 psi. When the distal portion of the first treatment member is greater than about 42,000 psi, the proximal portion of the first treatment member can have an elastic modulus in the range of about 1,500 psi to about 42,000 psi.

In a functional sense, in some embodiments of the double lumen gastrointestinal catheter, the distal portion of the first treatment member has a sufficiently high elasticity that upon insertion into the treatment site and release from a substantially linear constrained configuration, the distal end region assumes the preferred and anchoring configuration.

In some embodiments, the proximal portion of the first treatment member of the double lumen gastrointestinal catheter has an elastic modulus in the range of about 1,700 psi to about 25,000 psi. By way of example, a product of Arkema provides a polyether block amide product, PEBAX® 2533, having an elastic modulus of 1,750 psi and PEBAX® 633 having an elastic modulus of 41,300 psi. In a functional sense, in some embodiments of the double lumen gastrointestinal catheter, the proximal portion of the first treatment member comprises an elastic modulus that provides a level of compliance that is patient friendly by being more comfortable and less likely to cause irritation or injury.

In some embodiments, the distal end region of the first treatment catheter is substantially equivalent in length to the distal portion of the treatment catheter. In other embodiments, the distal end region of the first treatment catheter is less in length than the total length distal portion of the treatment catheter.

Various physical and structural properties of the first treatment member of the double lumen gastrointestinal catheter can underlie the difference in elastic modulus between the proximal and distal portions of the first treatment member. Accordingly, in some embodiments, the elasticity of the distal end region of the first treatment member is higher than that of the proximal portion by virtue of a difference in a material composition.

In some embodiments, the elasticity of the distal portion of the first treatment member is higher than that of the proximal portion by virtue of a difference in a sidewall structure of the first treatment member. For example, in some embodiments, the difference in a sidewall structure of the first treatment member includes a difference in thickness of the sidewall, as when the distal portion of the first treatment member includes a region of greater sidewall thickness than that of the proximal portion of the first treatment member. In some embodiments, the difference in a sidewall structure of the first treatment member includes a plurality of indents or inward axial convolutions in the sidewall of the proximal portion of the first treatment member, wherein an outer diameter of the indents or inward convolutions is less than an outer diameter of the distal portion of the first treatment member. In still further embodiments, the difference in a sidewall structure of the first treatment member includes variation in one or more layers of varying elastic modulus in the sidewall.

A first treatment site for which the double lumen gastrointestinal catheter is medically appropriate is typically an anatomical cavity, often the anatomical cavity is inflamed, infected, necrotizing, or otherwise abnormal or pathological. One particular example of such an anatomical cavity is an encapsulated or walled-off necrotic site involving the pancreas, wherein the distal end region of the treatment member is sized and configured for insertion into such an encapsulated pancreatic necrotic site.

A second treatment site for which the double lumen gastrointestinal catheter is medically appropriate is located in a small bowel or a stomach of the patient, or at any location in the gastrointestinal tract that is appropriate for enteral feeding or stomach-in fluid aspiration.

Some embodiments of the double lumen gastrointestinal catheter include a guidewire hosted within the lumen of the first treatment member. In a functional sense, this embodiment typically occurs as the first treatment member is passed over the guidewire, as the first treatment member is being advanced toward a treatment site. In particular embodiments, the guidewire is sufficiently stiff to maintain the distal end of the treatment member (in which it is disposed) in a substantially linear constrained configuration.

As noted above, in some embodiments, the preferred configuration of the distal end region of the first treatment member of the double lumen gastrointestinal catheter is one that anchors that region in the first treatment site, and accordingly, anchors the first treatment member, as a whole, therein. Merely by way of example, an anchoring configuration may take the form of a loop or multiple loops.

The functional purpose of the preferred configuration of the distal end region of the first treatment member is to anchor it in a first treatment site, such as, merely by way of example, a walled off necrotic site involving the pancreas. By the distal end region of the first treatment member anchoring in the first treatment site, the gastrointestinal catheter, as a whole, is anchored in the treatment site. By way of exemplifying an anchoring configuration, the preferred configuration of the distal end region of the distal portion of the treatment may be configured as a loop. In some embodiments, the preferred configuration includes two or more loops. _{∘}

It is also advantageous that the distal end of the first treatment member not injure the treatment site; accordingly, in some embodiments the distal end region of the first treatment member includes an atraumatic tip. In some embodiments, the distal end region of the first treatment member also includes a distal end fluid port in communication with the lumen.

Another aspect of the distal end region of the first treatment member embodiments of the double lumen gastrointestinal catheter relates to the fluid ports described to above. In some embodiments, the fluid ports are positioned within the distal end region of the first treatment member such that when the distal end is anchored within the first treatment site, the fluid ports are disposed in their substantial entirety within the treatment site.

Various aspects of the distal end region of the first treatment member of the double lumen gastrointestinal catheter relate to that region being formed, at least in part, by a shape memory composition. Accordingly, some embodiments of the gastrointestinal catheter have a higher level of stiffness in the distal portion of the first treatment member (compared to the proximal portion) that is at least partly attributable to a segment disposed within the distal portion having a shape memory composition. Some embodiments of the distal end region of the first treatment member have a segment that includes a shape memory composition, such as a shape memory polymer or a shape memory metal alloy. In some embodiments, the segment having a shape memory composition has a preferred configuration that is adapted to anchor the distal end of the treatment member within the treatment site.

By way of example, the shape memory composition may include nitinol in any appropriate form or configuration, such as wires in the form of any of a braid or axially-aligned linear wires. In some embodiments of the gastrointestinal catheter, the distal end region consists in its substantial entirety of nitinol. In some embodiments, the shape memory alloy segment is disposed within the lumen of the treatment member, against an inner surface of a treatment member sidewall. In some embodiments, the shape memory alloy segment is disposed within a sidewall of the treatment member, sandwiched between an inner and an outer polymer layer.

The dimensions of embodiments of the first treatment member of a double lumen gastrointestinal catheter be appropriate both for conveyance through an endoscope and appropriate for the anatomical dimensions of the routing to a treatment site. Accordingly, in some embodiments of the gastrointestinal catheter, the outer diameter of the first treatment member ranges from about 3 French to about 13 French. In some embodiments, the outer diameter of the first treatment member ranges from about 5 French to about 11 French. In typical embodiments, the length of the first treatment member is appropriate and sufficient to reach the treatment site by way of a route that originates at a percutaneous gastrostomy site.

In some embodiments of the double lumen gastrointestinal catheter, the proximal portions of the first and second treatment members are reversibly conjoinable by way of mutually engaging axially-aligned conjoining features. In some embodiments, the proximal portions of the first and second treatment members are conjoinable by way of both members being enclosed within a surrounding sheath. And in some embodiments, the proximal portions of the first and second treatment members are conjoinable but, when conjoined, the first and second treatment members are slidable with respect to each other.

In an embodiment of a double lumen gastrointestinal catheter, the first treatment member and the second treatment member are advanced into the patient’s body through the connection member and the percutaneous endoscopic gastrostomy (PEG) tube. When the first treatment member is advanced into the patient’s body, the second treatment member can be slidably advanced into the body relative to the first treatment member, or when the second treatment member is advanced into the patient’s body, the first treatment member may be slidably advanced into the patient’s body relative to the second treatment member. When both the first treatment member and the second treatment member are in the patient’s body, the first treatment member and the second treatment member are detachably or slidably conjoinable side by side at their proximal portions, and the first treatment member and the second treatment member can be pulled out of patient’s body metachronously.

One embodiment of the disclosure is in the form of a kit for a double lumen gastrointestinal catheter. Embodiments of the kit include a double lumen gastrointestinal catheter, as described above, and a guidewire sized and configured to be hosted within the lumen of the first treatment member, the guidewire having a distal tip, the guidewire having sufficient length that its distal tip can extend beyond the distal end region of the treatment member.

### Embodiment 4 Method: Flushing a First Treatment Site, and Enteral Feeding at a Second Treatment Site or Aspirating Fluid at a Second Treatment Site

Embodiments of the disclosure include a method of flushing a first treatment site and feeding a patient directly at a second treatment site with a single percutaneously-implanted double lumen gastrointestinal catheter. Accordingly, the method includes placing a percutaneous endoscopic gastrostomy (PEG) tube in a patient thereby providing an entry route for the double lumen gastrointestinal catheter into the patient’s gastrointestinal tract, the catheter having (1) a first treatment member comprising a first lumen and (2) a second treatment member comprising a second lumen. The method further includes distally advancing the second treatment member into a second treatment site in a small bowel or a stomach of the patient; and distally advancing the first treatment member over a guidewire to a position such that a distal end region of the first treatment member is positioned within a first treatment site. Embodiments of the method include advancing the first treatment member before advancing the second treatment member; that sequence, however, may risk dislodgement of the first treatment member during manipulations of the second treatment member. Accordingly, typical embodiments of the method involve placement of the second treatment member at the second treatment site prior to placement of the first treatment member at the first treatment site.

The method further includes withdrawing the guidewire proximally from the first treatment member, thereby anchoring the distal end region of the first treatment member within the first treatment site. The method further includes flowing an irrigation fluid through the first treatment member and into the first treatment site; and further still, flowing a solution through the second treatment member into the second treatment site or aspirating a fluid at the second treatment site through the second treatment member. With respect to the treatment sites, the first treatment site, such as a walled off necrotic site involving the pancreas, is in medical need of being flushed or irrigated with a fluid and the second treatment site is an appropriate site for enteral feeding of the patient.

Some embodiments of the method further include positioning an endoscope in the gastrointestinal tract of the patient to illuminate and allow visualization of the operating field, and to assist in the placement of treatment members of the double lumen gastrointestinal catheter. Accordingly, distally advancing the second treatment member may include towing the second treatment member with the endoscope, as enabled by forceps, to the second treatment site, and distally advancing the first treatment may include towing the first treatment member with the endoscope to a position proximate an entry route into the first treatment site.

Some embodiments of the method are implemented with a double lumen gastrointestinal catheter in which the distal end region of the first treatment member has a preferred anchoring configuration but is also capable of elastically assuming a constrained linear configuration when the guidewire is disposed within the lumen of the first treatment member. Accordingly, in some embodiments of the method, anchoring the distal end region of the first treatment member includes withdrawing the guidewire from the distal end region thereby allowing the distal end region to reconfigure into its preferred anchoring configuration. In particular embodiments of the of the double lumen gastrointestinal catheter, the distal end region of first treatment member includes a shape memory segment; in these embodiments, reconfiguring into the preferred configuration entails the shape memory segment reconfiguring from a constrained linear configuration to its preferred configuration.

In some embodiments of the method, prior to distally advancing the first treatment member over the guidewire, the method includes distally advancing a guidewire through the PEG tube until a distal tip of the guidewire is situated within the treatment site.

In practicing an embodiment of the method, entry of the distal end of the first treatment member into the first treatment site may be difficult. In such an incident, the method (prior to distally advancing the endoscope to a position such that the distal end of the endoscope is positioned proximate an entry route into the treatment site) may include placing a stent within the entry route into the first treatment site, and thus advancing the first treatment member distally from the endoscope may include advancing the treatment member through an opening provided by the stent.

In addition to flowing a fluid into the first treatment site, as above, embodiments of the method may include removing fluid from the first treatment site. Accordingly, some embodiments of the method include draining fluid from the first treatment site. Draining fluid may further include removing cellular debris from the treatment site. Draining fluid from the treatment site may include both (a) draining an irrigation fluid that has been injected into the site as well as (b) draining a fluid from any other source within the treatment site. In particular embodiments of the method, draining fluid may include withdrawing a fluid from the first treatment site through fluid ports disposed within the distal end region of the first treatment member. Draining fluid from the treatment site may be facilitated by a vacuum or suction-based draw of fluid in a distal to proximal path.

Some embodiments of the method of flushing a first treatment site, as above, may include mixing an active agent into the irrigation fluid prior to the flowing the irrigation fluid into the first treatment site. By way of example, an active agent may include any of an anti-infectious agent, or any pharmaceutically effective agent. In some embodiments, the active agent may be considered to be substantially inert, pharmaceutically, but serve another medical purpose, such as may be provided by an imaging compound.

With respect to flowing a solution through the second treatment member into a second treatment site, such solution is typically an enteral feeding solution. In some embodiments, the solution may include a pharmaceutically effective agent.

With respect to aspirating a fluid at the second treatment site through the second treatment member, an embodiment of the method includes aspirating a fluid, by the second treatment member, from a patient’s stomach. The fluid may include an irrigation fluid that has been injected into the first treatment site and drained therefrom and collected in the stomach. Correspondingly, aspirating the fluid by the second treatment member may include aspirating, by the second treatment member, the irrigation fluid that has been injected into the first treatment site and drained therefrom and collected in the stomach. The ability to aspirate through the second treatment member may prevent reflux of fluid contents collecting in the stomach, or solely for decompression of the stomach on an ‘as needed’ basis (can be activated when needed or desired).

In typical embodiments of the method, involving implementation of treatment at the first and second treatment site, the treatment needs to occur over a treatment period, as determined by a physician for a treatment duration that may range upward toward several weeks. Accordingly, in some embodiments of the methods the first and second treatment members remain in place the first and second treatment sites, respectively, over the duration of a treatment period. And upon completion of a medically appropriate duration, the method concludes by withdrawing the treatment member from the treatment site.

### Embodiment 4 Method: Treating Gastrointestinal Treatment Sites

In another aspect, embodiments of the disclosure include a method of treating treatment sites in a gastrointestinal tract with a percutaneously-implanted double lumen gastrointestinal catheter. The method includes placing a percutaneous endoscopic gastrostomy (PEG) tube in a patient thereby providing an entry route for the double lumen gastrointestinal catheter into the patient’s gastrointestinal tract, the catheter having (1) a first treatment member comprising a first lumen and (2) a second treatment member comprising a second lumen. The method includes distally advancing the second treatment member into a second treatment site in a small bowel or a stomach of the patient. The method further includes distally advancing the first treatment member to a position such that a distal end region of the first treatment member is positioned within a first treatment site, and then anchoring the distal end region of the first treatment member within the first treatment site. Some embodiments of the method involve placement of the second treatment member at the second treatment site prior to placement of the first treatment member at the first treatment site. The method further includes flowing an irrigation fluid through the first treatment member and into the first treatment site; and further still, flowing a solution through the second treatment member into the second treatment site or aspirating a fluid at the second treatment site through the second treatment member. With respect to the treatment sites, the first treatment site, such as a walled off necrotic site involving the pancreas, is in medical need of being flushed or irrigated with a fluid and the second treatment site is an appropriate site for enteral feeding of the patient or aspirating fluid in the stomach of the patient.

### Embodiment 4 Method: Treating Gastrointestinal Treatment Sites

In a further aspect, embodiments of the disclosure include a method of flushing a first treatment site and feeding a patient directly at a second treatment site with a single percutaneously-implanted double lumen gastrointestinal catheter. Accordingly, the method includes placing a percutaneous endoscopic gastrostomy (PEG) tube in a patient thereby providing an entry route for the double lumen gastrointestinal catheter into the patient’s gastrointestinal tract, the catheter having (1) a first treatment member comprising a first lumen and (2) a second treatment member comprising a second lumen. The method further includes distally advancing the second treatment member into a second treatment site in a small bowel of the patient; and distally advancing the first treatment member over a guidewire to a position such that a distal end region of the first treatment member is positioned within a first treatment site, the guidewire constraining the distal end region in a substantially linear configuration. The method further releasing the distal end region from the guidewire constraint, thereby allowing the distal end region to assume an anchoring configuration. The method further includes flowing an irrigation fluid through the first treatment member and into the first treatment site; and further still, flowing a solution through the second treatment member into the second treatment site.

In a further aspect, embodiments of the disclosure include a method of flushing a first treatment site and aspirating a fluid at the second treatment site through the second treatment member with a single percutaneously-implanted double lumen gastrointestinal catheter. Accordingly, the method includes placing a percutaneous endoscopic gastrostomy (PEG) tube in a patient thereby providing an entry route for the double lumen gastrointestinal catheter into the patient’s gastrointestinal tract, the catheter having (1) a first treatment member comprising a first lumen and (2) a second treatment member comprising a second lumen. The method further includes distally advancing the second treatment member into a second treatment site in a stomach of the patient; and distally advancing the first treatment member over a guidewire to a position such that a distal end region of the first treatment member is positioned within a first treatment site, the guidewire constraining the distal end region in a substantially linear configuration. The method further releasing the distal end region from the guidewire constraint, thereby allowing the distal end region to assume an anchoring configuration. The method further includes flowing an irrigation fluid through the first treatment member and into the first treatment site; and further still, aspirating the fluid at the second treatment site through the second treatment member.

### Embodiment 4 Method: Treating Gastrointestinal Treatment Sites

An embodiment of the present disclosure includes a method of flushing a first treatment site and feeding a patient directly at a second treatment site with a percutaneously-implanted double lumen gastrointestinal catheter. The method includes placing a percutaneous endoscopic gastrostomy (PEG) tube in a patient’s body, and arranging a connection member on a side of the PEG tube outside the patient’s body, so as to provide an entrance route for the gastrointestinal catheter to enter the patient’s gastrointestinal tract, wherein the gastrointestinal catheter includes a first treatment member and a second treatment member; distally advancing the second treatment member to the second treatment site in the patient’s small bowel or the stomach through the second connection end of the connection member, and connecting the second connection end of the connection member with the second treatment member through the second sealing cap and a corresponding sealing part; advancing the guidewire through the first connection end of the connecting member distally to the first treatment site; after the distal end region of the distal portion of the first treatment member is straightened using a straight rectifying tube, distally advancing the straightened first treatment member over the guidewire, where the straight rectifying tube is disposed on the first treatment member via its seam on its side wall and is used to straighten the first treatment member by advancing the straight rectifying tube on the first treatment member, and before the first treatment member is advanced into the patient’s body, advancing the straight rectifying tube independently distally relative to the first treatment member to make the entire straight rectifying tube be located around the guidewire, then removing the straight rectifying tube along its seam from the guidewire; advancing the distal end region of the first treatment member distally to the first treatment site.

The method further includes, after the distal end region of the first treatment member is advanced to the first treatment site, pulling out the guidewire from the patient’s body, so that the distal end region of the first treatment member can be released from a substantial linear constrained configuration; pulling the traction wire at the proximal portion of the first treatment member, to enable the distal end region of the first treatment member to assume a locked loop-like configuration, so that the distal end region may be anchored at the first treatment site; connecting the first connection end of the connection member with the first treatment member through the first sealing cap and the corresponding sealing part; flushing the first treatment member with a solution flowing through the first treatment member and into the first treatment site; flowing a solution through the second treatment member and entering the second treatment site to feed the bowel or aspirating a fluid in the stomach of the second treatment site through the second treatment member; connecting the third sealing cap with an end port of the proximal portion of the first treatment member.

Any one or more features or steps of any embodiment of the disclosures disclosed herein (device or method) can be combined with any one or more other features of any other embodiment of the disclosures, without departing from the scope of the disclosure. It should also be understood that the disclosures are not limited to the embodiments that are described or depicted herein for purposes of exemplification, but are to be defined only by a fair reading of claims appended to the patent application, including the full range of equivalency to which each element thereof is entitled.

What is claimed is:

1. A gastrointestinal catheter for insertion into a treatment site in a patient, the site in need of medical treatment, the treatment site comprising at least one of a first treatment site and a second treatment site, the catheter comprising:
a treatment member, the treatment member comprising at least one of a first treatment member and a second treatment member and comprising a lumen, a proximal portion, a distal portion, and a distal end region within the distal portion;
wherein the first treatment member is sized and configured for insertion of its distal end into the first treatment site in an upper portion of a gastrointestinal tract, and wherein the second treatment member is sized and configured for placement of its distal end into the second treatment site within a bowel or a stomach of the patient;
wherein the distal end region of the first treatment member comprises a anchoring configuration, the treatment member being capable of assuming a linear constrained configuration, wherein the anchoring configuration is adapted to anchor the distal end region within the first treatment site;
wherein the distal end region of the first treatment member comprises one or more fluid ports in communication with the lumen, wherein the fluid ports are configured to allow through flow of a fluid;
wherein the proximal portions of the first and second treatment members are detachably or slidably conjoinable side by side, and wherein the distal portions of the first and second treatment members are separated from each other.

2. The gastrointestinal catheter of claim 1, wherein the distal end region of the first treatment member comprises a sufficiently high elasticity that upon insertion into the first treatment site and release from the linear constrained configuration, the distal end region assumes the anchoring configuration.

3. The gastrointestinal catheter of claim 1, wherein the proximal portion of the first treatment member comprises an elastic modulus that provides a level of compliance that mitigates a possibility of injury to the patient in a facial area proximate a nasal site of entry of the first treatment member.

4. The gastrointestinal catheter of claim 1, wherein the elasticity of the distal end region of the first treatment member is not less than that of the proximal portion by virtue of a proximal-distal difference in material composition or a difference in a sidewall structure of the first treatment member.

5. The gastrointestinal catheter of claim 4, wherein the difference in a sidewall structure of the first treatment member comprises a thickness of the sidewall, and wherein the distal portion of the first treatment member comprises a region of greater sidewall thickness than that of the proximal portion of the first treatment member; or
wherein the difference in a sidewall structure of the first treatment member comprises a plurality of indents or inward axial convolutions in the sidewall of the proximal portion of the first treatment member, wherein an outer diameter of the indents or inward axial convolutions is not greater than an outer diameter of the distal portion of the first treatment member; or
wherein the difference in a sidewall structure of the first treatment member comprises variation in one or more layers of varying elastic modulus in the sidewall.

6. The gastrointestinal catheter of claim 1, wherein the first treatment site comprises an anatomical cavity, and the anatomical cavity is in need of medical treatment because of infection and/or necrosis.

7. The gastrointestinal catheter of claim 6, wherein the anatomical cavity is an encapsulated pancreatic necrotic site, and wherein the distal end region of the first treatment member is sized and configured for insertion into the encapsulated pancreatic necrotic site.

8. The gastrointestinal catheter of claim 1, further comprising a guidewire hosted within the lumen of the at least one of the first treatment member and the second treatment member;
wherein the guidewire is sufficiently stiff to maintain the distal end of the first treatment member in the linear constrained configuration.

9. The gastrointestinal catheter of claim 1, further comprising a connection member, wherein the connection member comprises three connection ends, wherein one connection end is connected with a PEG tube, and the other two connection ends are a first connection end and a second connection end respectively; wherein the first connection end is fixed with the first treatment member and the second connection end is fixed with the second treatment member.

10. The gastrointestinal catheter of claim 1, wherein the anchoring configuration of the distal end region of the distal portion of the first treatment member comprises a at least one loop adapted to anchor within the first treatment site.

11. The gastrointestinal catheter of claim 1, wherein the distal end region of the first treatment member comprises an atraumatic tip or a distal end fluid port in communication with the lumen.

12. The gastrointestinal catheter of claim 1, wherein the fluid ports are positioned within the distal end region of the first treatment member such that when the distal end is anchored within the first treatment site, the fluid ports are disposed within the first treatment site.

13. The gastrointestinal catheter of claim 1, wherein the distal end region of the first treatment member comprises a segment comprising a shape memory composition or the first treatment member comprises a traction wire for drawing the distal end region thereof to make the distal end region of the first treatment member assumes the anchoring configuration;
wherein the shape memory composition comprises any of a shape memory polymer or a shape memory metal alloy; wherein the segment comprising a shape memory composition comprises the anchoring configuration adapted to anchor the distal end of the treatment member within the treatment site.

14. The gastrointestinal catheter of claim 13, wherein the shape memory alloy segment is disposed within the lumen of the treatment member, against an inner surface of a treatment member sidewall, or
wherein the shape memory alloy segment is disposed within a sidewall inner the treatment member, sandwiched between an inner and an outer polymer layer.

15. The gastrointestinal catheter of claim 1, wherein the outer diameter of the first treatment member ranges from 3 French to 13 French.

16. The gastrointestinal catheter of claim 1, wherein the length of the at least one of the first treatment member and the second treatment member is/are sufficient to reach the treatment site by way of a route that originates in the nostril or at a percutaneous gastrostomy site of the patient.

17. The gastrointestinal catheter of claim 1, wherein the proximal portions of the first and second treatment members are conjoinable by way of both members being enclosed within a surrounding sheath.

18. A gastrointestinal catheter kit, comprising:
the gastrointestinal catheter of claim 1; and
a pusher member sized and configured to fit within the endoscope, the pusher member comprising a lumen, wherein the pusher member is configured to stabilize a position of the gastrointestinal catheter in at least one of the first treatment site and the second treatment site.

19. The gastrointestinal catheter kit of claim 18, further comprising:
a guidewire sized and configured to be hosted within the lumens of both the pusher member and the first treatment member, the guidewire comprising a distal tip, the guidewire comprising sufficient length that its distal tip extends beyond the distal end region of the treatment member.

20. A method of flushing a first treatment site with the gastrointestinal catheter of claim 1, comprising:
placing a percutaneous endoscopic gastrostomy (PEG) tube in the patient thereby providing an entry route for the at least one of the first treatment member and the second treatment member into a gastrointestinal tract of the patient;
advancing the second treatment member distally into a second treatment site in a small bowel or a stomach of the patient;
advancing the first treatment member over a guidewire to a position such that a distal end region of the first treatment member is positioned within a first treatment site;
withdrawing the guidewire proximally from the first treatment member, thereby anchoring the distal end region of the first treatment member within the first treatment site;
flowing an irrigation fluid through the treatment member and into the treatment site to flush the first treatment site; and
flowing a solution through the second treatment member into the second treatment site to conduct enteral feeding or aspirating a fluid at the second treatment site through the second treatment member.

21. The method of claim 20, wherein before advancing the first treatment member over a guidewire, the method further comprises using a straight rectifying tube to straighten the distal end region of the first treatment member.

22. The method of claim 20, further comprising positioning an endoscope in the gastrointestinal tract of the patient, and wherein distally advancing the second treatment member comprises towing the second treatment member with the endoscope to the second treatment site, and wherein distally advancing the first treatment comprises towing the first treatment member with the endoscope to a position proximate an entry route into the first treatment site.

23. The method of claim 20, wherein the distal end region of the first treatment member comprises a anchoring configuration but is also capable of elastically assuming a constrained linear configuration when the guidewire is disposed within the first treatment member, and wherein anchoring the distal end region of the first treatment member comprises withdrawing the guidewire from the distal end region thereby allowing the distal end region to reconfigure into its anchoring configuration.

24. The method of claim 23, wherein the distal end region of the first treatment member comprises a segment comprising a shape memory composition, and wherein the distal end region reconfiguring into the anchoring configuration comprises the shape memory segment reconfiguring from the constrained linear configuration to the anchoring configuration; or the first treatment member comprises a traction wire, and wherein the distal end region reconfiguring into the anchoring configuration comprises drawing the distal end region with the traction wire to form the anchoring configuration.

25. The method of claim 20, wherein prior to distally advancing the first treatment member over the guidewire, the method comprises distally advancing guidewire through the PEG tube until a distal tip of the guidewire is situated within the first treatment site.

26. The method of claim 20, further comprising draining fluid from the first treatment site by way of a lumen in the first treatment member, wherein draining fluid from the first treatment site further comprises draining an irrigation fluid that has been injected into the site as well as a fluid from any other source within the treatment site.

27. The method of claim 26, wherein draining fluid comprises withdrawing a fluid from the first treatment site through fluid ports within the distal end region of the first treatment member.

28. The method of claim 20, further comprising mixing an active agent into the irrigation fluid prior to the flowing the irrigation fluid into the first treatment site.

29. The method of claim 20, wherein the solution flowing through the second treatment member comprises an enteral feeding solution.

30. The method of claim 20, wherein the first and second treatment members remain at the first and second treatment sites, respectively, for a treatment period, and wherein upon completion of a treatment period, the method comprises withdrawing the first treatment member from the first treatment site and withdrawing the second treatment member from the second treatment site metachronously.

31. A method of treating treatment sites in a gastrointestinal tract with the gastrointestinal catheter of claim 1, the method comprising:
placing a percutaneous endoscopic gastrostomy (PEG) tube in a patient thereby providing an entry route for the double lumen gastrointestinal catheter into a gastrointestinal tract of the patient, the catheter comprising (1) a first treatment member comprising a first lumen and (2) a second treatment member comprising a second lumen;
distally advancing the second treatment member into a second treatment site in a small bowel or a stomach of the patient;
distally advancing the first treatment member to a position such that a distal end region of the first treatment member is positioned within a first treatment site;
anchoring the distal end region of the first treatment member within the first treatment site;
flowing an irrigation fluid through the first treatment member and into the first treatment site to flush the first treatment site; and
flowing a solution through the second treatment member into the second treatment site to conduct enteral feeding or aspirating a fluid at the second treatment site through the second treatment member.
